(19) United States
(12) Patent Application Publication
(10) Pub. No.: US 2026/0258472 A1
(43) Pub. Date: Sep. 3, 2026

(54) METHODS FOR DETECTING THE PRESENCE OF AT LEAST ONE MISFOLDED FORM OF SOD1 IN A BIOLOGICAL SAMPLE

(71) Applicant: Mclaughlin Research Institute for Biomedical Sciences, Great Falls, MT (US)

(72) Inventor: Moses LEAVENS, Great Falls, MT (US)

(21) Appl. No.: 19/489,459

(22) PCT Filed: Jun. 5, 2024
(86) PCT No.: PCT/US2024/032553

§ 371 Date: Dec. 2, 2025

Related U.S. Application Data
(60) Provisional application No. 63/506,405, filed on Jun. 6, 2023.
(60) Provisional application No. 63/625,611, filed on Jan. 26, 2024.

(51) Int. Cl. C12Q 1/26

(52) U.S. Cl. CPC C12Q 1/26; C12Y 115/01001; G01N 2800/2821; G01N 2800/2835; G01N 2800/52

(57) ABSTRACT

Disclosed herein are methods for detecting the presence of at least one misfolded form of human Superoxide Dismutase 1 (SOD1) in a biological sample obtained from a human subject. In some aspects, the subject is suspected of having, or has, one or more neurodegenerative diseases, such as, for example, Amyotrophic Lateral Sclerosis, Parkinson's disease, or Alzheimer's disease.

### RELATED APPLICATION INFORMATION

This application claims priority to U.S. Application No. 63/506,405, filed on Jun. 6, 2023, and U.S. Application No. 63/625,611, filed on Jan. 26, 2024, the entire contents of each of which are herein incorporated by reference.

### STATEMENT REGARDING FEDERALLY SPONSORED RESEARCH OR DEVELOPMENT

None.

### SEQUENCE LISTING STATEMENT

The contents of the electronic sequence listing titled MCLA-42129-601-ST26.xml (Size: 19,903 bytes; and Date of Creation: Jun. 5, 2024) is herein incorporated by reference in its entirety.

### TECHNICAL FIELD

The present disclosure relates to methods for detecting the presence of at least one misfolded form of human Superoxide Dismutase 1 (SOD1) in a biological sample obtained from a human subject. In some aspects, the subject is suspected of having, or has, one or more neurodegenerative diseases, such as, for example, Amyotrophic Lateral Sclerosis, Parkinson's disease or Alzheimer's disease.

### BACKGROUND

Amyotrophic Lateral Sclerosis (ALS) is a rapidly progressive neuromuscular disease where motor neurons degenerate in patients, leaving them with an inability to innervate muscle, where patients typically succumb to this disease from respiratory failure (https://doi.org/10.1016/j.chest.2018.06.035). The initial symptoms of this rapidly progressive neurodegenerative disease include fasciculations and muscle fatigue, with more advanced symptoms including weight loss, paralysis, a tracheotomy, and loss of speech (https://doi.org/10.1111/ene.14393, https://doi.org/10.1111/ene.14393). Most ALS cases are sporadic (sALS), which accounts for approximately 90% of ALS cases, while approximately 10% of ALS cases develop because of identified genetic aberrations that are inherited. The primary inherited genetic aberration in ALS is abnormal hexanucleotide repeat expansions in the intronic region of Chromosome 9 open reading frame 72 (C9ORF72) (https://doi.org/10.1016/j.neuron.2011.09.011,DOI: 10.1016/j.neuron.2011.09.010, https://doi.org/10.1016/S1474-4422 (11) 70261-7). Other types of inherited or familial ALS (fALS) include autosomal dominant mutations in the gene Superoxide Dismutase 1 (SOD1), which accounts for about 20% of familial ALS and 2% of all ALS (https://doi.org/10.1038/s41598-021-03891-8, https://doi.org/10.1016/j.jmb.2020.09.025). Several autosomal dominant mutations in SOD1 cause the SOD1 protein to fold abnormally (https://doi.org/10.1016/j.jmb.2020.09.025), where this abnormal folding of SOD1 is thought to be a toxic gain of function containing prion properties (https://doi.org/10.1002/ana.21319, https://doi.org/10.3390/ijms22084155). While SOD1 misfolding and prion spreading occurs in SOD1 fALS (https://doi.org/10.1007/s00401-016-1623-4), less is known about non-mutant/wild type SOD1 misfolding in sporadic ALS patients, which may be involved in the pathology of sALS (https://doi.org/10.1038/s41598-018-31773-z) and C9ORF72 familial ALS (https://doi.org/10.1136%2Fjnnp-2018-319386).

Few ALS biomarkers can measure the misfolded proteins at low dilutions that are thought to propagate and spread in people affected with ALS. Diagnosing ALS takes approximately 1 year and on some occasions can be misdiagnosed (https://doi.org/10.1016/j.clineuro.2009.10.014). Thus, reliable biomarkers that can detect ALS early and accurately are needed to improve diagnosis of ALS, improve clinical trials for ALS, and monitor disease progression

### SUMMARY

In a first embodiment, the present disclosure relates to a method of preparing a purified human Superoxide Dismutase 1 (SOD1) substrate for use in a real-time quaking induced conversion assay. The method comprises:
a. performing chromatography on a sample of cells expressing a human SOD1 protein having an amino acid sequence of SEQ ID NO:1 or SEQ ID NO:2, to produce an eluate; and
b. either:
   i. incubating the eluate for at least 6 hours at a temperature of about 20° C. to about 22° C. in at least 20 mM to about 30 mM of at least one buffer having a pH between about 6.0 to about 8.0 to produce a purified human SOD1 substrate; or
   ii. performing, in the presence of at least 20 mM to about 30 mM of at least one buffer having a pH between about 6.0 to about 8.0, buffer exchange, exchange using an exclusion column which removes any salts and/or contaminants less than 10 kDa, or dialysis for at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours to produce a purified human SOD1 substrate.

In some aspects of the above method, the method further comprises: (a) centrifuging the eluate after incubation to obtain a supernatant; and (b) filtering the supernatant to produce a purified human SOD1 substrate.

In another aspect of the above method, the chromatography is liquid chromatography, ion exchange chromatography, affinity chromatography, size exclusion chromatography, or any combinations thereof.

In still another aspect of the above method, the buffer is a Tris buffer, a sodium phosphate buffer, HEPES, Tris-HCl, or any combinations thereof.

In still yet a further aspect of the above method, the eluate is incubated for at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

In still yet a further aspect of the above method, the concentration of at least one buffer is between about 10 mM to about 90 mM.

In still yet a further aspect of the above method, the pH is about 8.0.

In still yet another aspect of the above method, the (a) buffer is a Tris buffer having a pH of 8.0; (b) the concentration of the Tris buffer is 25 mM; (c) and the temperature is 20° C.

In a second embodiment, the present disclosure relates to a method for detecting the presence of at least one misfolded form of human Superoxide Dismutase 1 (SOD1) in a biological sample obtained from a human subject. The method comprises the steps of:
a. performing a real-time quaking induced conversion (RT-QuIC) assay or seed amplification assay (SAA) on a biological sample obtained from a subject suspected of having a neurodegenerative disease, wherein the RT-QuIC assay:
i. uses the human SOD1 substrate produced according to the method of the first embodiment; and
ii. is performed:
1. using a buffer comprising: (i) about 0.5 M to about 0.75 M guanidine HCl or about 0.5 M to about 0.75 M urea; and (ii) about 0.01 M to about 0.05 M sodium acetate;
2. using at least one fluorescent compound;
3. at a pH of about 3.5 to about 4.5;
4. at a shaker speed of about 300 to about 700 rpm; and
5. at a temperature of about 35° C. to about 42° C.; and
b. detecting the presence of the at least one fluorescent compound, wherein the detection of the presence of the at least one fluorescent compound indicates the presence of at least one misfolded form of SOD1 in the biological sample.

In one aspect of the above method, the buffer comprises about 0.5 M to about 0.75 M guanidine HCl. In another aspect of the above method, the buffer comprises about 0.5 M to about 0.75 M urea.

In yet another aspect of the above method, the buffer further comprises EDTA, one or more reducing agents, one or more salts, or any combination thereof.

In another aspect of the above method, the concentration of human SOD1 substrate used in the assay is from about 10 μM to about 60 μM. In another aspect of the above method, In another aspect of the above method, the concentration of human SOD1 substrate used in the assay is about 10 μM. In another aspect of the above method, the concentration of human SOD1 substrate used in the assay is about 20 μM. In another aspect of the above method, In another aspect of the above method, the concentration of human SOD1 substrate used in the assay is about 30 μM. In another aspect of the above method, In another aspect of the above method, the concentration of human SOD1 substrate used in the assay is about 40 μM. In another aspect of the above method, In another aspect of the above method, the concentration of human SOD1 substrate used in the assay is about 50 μM. In another aspect of the above method, In another aspect of the above method, the concentration of human SOD1 substrate used in the assay is about 60 μM.

In still yet another aspect of the above method, the concentration of SOD1 substrate used in the assay is about 30 μM based on an extinction coefficient of 5,500 M⁻¹ cm⁻¹. In still yet another aspect of the above method, the concentration of SOD1 substrate used in the assay is about 50 μM based on an extinction coefficient of 5,500 M⁻¹ cm⁻¹.

In still yet another aspect of the above method, the buffer comprises (i) about 0.6 M guanidine HCl or about 0.6 M urea; and (ii) about 0.02 M sodium acetate.

In still yet a further aspect of the above method, the buffer comprises (i) about 0.6 M guanidine HCl; and (ii) about 0.02 M sodium acetate.

In still yet another aspect of the above method, the buffer comprises (i) about 0.6 M urea; and (ii) about 0.02 M sodium acetate.

In still yet another aspect of the above method, the buffer further comprises EDTA, one or more reducing agents, and one or more salts. In some aspects, the one or more reducing agents is β-mercaptoethanol (βME), Dithiothreitol (DTT), tris(2-carboxyethyl)phosphine (TCEP). In yet further aspects, the reducing agent is βME. The one or more salts can be sodium chloride, potassium chloride or calcium chloride. In some aspects, the one or more salts are sodium chloride.

In yet further aspects, the buffer further comprises EDTA, βME and sodium chloride.

In still yet another aspect of the above method, the pH is about 4.0.

In still yet another aspect of the above method, the shaker speed is about 500 rpm.

In still yet a further aspect of the above method, the temperature is about 37° C.

In still yet another aspect of the above method, the shaker speed is about 500 rpm and the temperature is about 37° C.

In still yet a further aspect of the above method, the fluorescent compound is an amyloid binding dye. In some aspects, the concentration of the fluorescent compound used in the assay is from about 5 μM to about 100 μM. In some aspects, the concentration fluorescent compound is 20 μM. In some aspects, the fluorescent compound is Thioflavin T. In yet further aspects, the fluorescent compound is Thioflavin T and the concentration of Thioflavin T used in the assay is 20 μM.

In still yet a further aspect of the above method, the RT-QuIC assay is performed:
1. using a buffer comprising: (i) about 0.6 M guanidine HCl; and (ii) about 0.02 M sodium acetate;
2. using at least Thioflavin T as the fluorescent compound;
3. at a pH of about 4.0;
4. at a shaker speed of about 500 rpm; and
5. at a temperature of about 37° C.

In still yet a further aspect of the above method, the RT-QuIC assay is performed:
1. using a buffer comprising: (i) about 0.6 M guanidine HCl; (ii) about 0.02 M sodium acetate; (iii) EDTA; (iv) βME; and (v) sodium chloride.
2. using at least Thioflavin T as the fluorescent compound;
3. at a pH of about 4.0;
4. at a shaker speed of about 500 rpm; and
5. at a temperature of about 37° C.

In yet a further aspect of the above method, the RT-QuIC assay employs one or more solid supports, such as one or more beads or a 96-well plate or 384-well plate.

In some aspects of the above method, the biological sample is cerebrospinal fluid, tissue, whole blood, serum, plasma, saliva, nasal brushings, skin, urine, tears, or any combinations thereof. In some aspects, the biological sample is cerebrospinal fluid. In some aspects, the biological sample is tissue, such as spinal cord tissue, brain tissue, or any combinations thereof. In yet further aspects, the biological sample is whole blood. In yet other aspects, the biological sample is serum. In still further aspects, the biological sample is plasma. In still further aspects, the biological sample is saliva. In still yet further aspects, the biological sample is nasal brushings. In still yet further aspects, the biological sample is urine. In yet further aspects, the biological sample is tears.

In yet further aspects of the above method, the neurodegenerative disease is amyotrophic lateral sclerosis (ALS), Parkinson's disease or Alzheimer's disease. In some aspects, the neurodegenerative disease is ALS; the ALS can be familial ALS or sporadic ALS.

In yet a further aspect of the above method, the method further comprises diagnosing a subject as having a neurodegenerative disease based on the detection of the presence of one or more misfolded forms of SOD1 in the biological sample obtained from the human subject.

In some aspects, the method further comprises monitoring the subject for progression of the neurogenerative disease.

In some aspects, the method further comprises administering one or more treatments to the subject diagnosed as having a neurodegenerative disease (e.g., ALS, Parkinson's disease, or Alzheimer's disease).

In further aspects, the assay uses one or more solid supports. In some aspects, the solid support is one or more beads. In still other aspects, the solid support is a 96-well plate or 384-well plate.

### BRIEF DESCRIPTION OF THE DRAWINGS

FIG. 1 shows a cDNA sequence (SEQ ID NO:3) of the human SOD1 substrate, the corresponding amino acid sequence with an underlined Histag adjacent to thrombin tag (SEQ ID NO: 1) and an amino acid sequence of the human SOD1 substrate without the underlined Histag adjacent to thrombin tag (SEQ ID NO:2).

FIG. 2 shows synthetic aggregates/seeds of human SOD1 substrate detected by SOD1 RT-QuIC at multiple 10-fold dilutions.

FIG. 3 shows an SOD1 RT-QuIC assay detecting and propagating misfolded SOD1 protein in a human SOD1 familial ALS (SOD1 fALS) patient as well as two human sALS spinal cords at 10⁻³ tissue dilutions with 100% sensitivity and 100% specificity.

FIG. 4 shows misfolded SOD1 protein detected and propagated in two human Chromosome 9 open reading frame 72 familial ALS (C9ORF72 fALS) patients at 10⁻³ spinal cord dilutions using the SOD1 RT-QuIC assay, with 100% sensitivity and 100% specificity.

FIG. 5 shows SOD1 seeding activity in two human idiopathic Parkinson disease brains (orange and green) versus a negative control (black) at 104 and 10⁻⁵ tissue dilutions.

FIG. 6 shows in the left panel, fast protein liquid chromatogram of BL21 E. coli expressed human SOD1 WT eluted via an imidazole gradient under reducing conditions. The later eluting peak, peak 2 (pk2, 120-140 mL), is human SOD1, determined via SDS-PAGE (no heat, no reducing agent) stained with Coomassie (right panel), showing pk2 is in dimer and monomer equilibrium.

FIG. 7 shows Native PAGE immunoblot of T7 or BL21 E. coli expressed human SOD1 purified with reducing agent that is subsequently exchanged into 25 mM Tris pH 8.0 (1 or 3 μg lanes). For BL21 E. coli expressed human SOD1 WT, exchange from 25 mM Tris pH 8.0 into RTQuIC buffer shifts equilibrium from dimer/monomer population to primarily monomer population. Dimer is represented as ‘D’ and monomer is represented as ‘M’. Reduced treated human SOD1 in the RTQuIC buffer was used as a substrate for RT-QuIC (RTQuIC lane).

FIG. 8 shows liquid chromatography electrospray ionization mass spectrum (top) and deconvoluted mass spectrum (bottom) of purified human SOD1, with experimental mass of 17,941.9892 atomic mass unit (amu). The expected mass is 17,867.86 amu (from ExPASy Peptide Mass tool (http://web.expasy.org/peptide mass/) using average mass, with no cutting and [M+H]⁺ options. The amu difference could be attributable to Methionine side-chain mass (75 Da) loss on N-terminus of SEQ ID NO: 1.

FIG. 9 shows far UV CD spectroscopy of 50 μM human SOD1 WT purified in the presence of a reducing agent (—SH) or not purified with a reducing agent (oxidized, S—S). Reduced human SOD1 was exchanged into a Tris buffer pH 8.0 (See, FIG. 7) then put in RTQuIC buffer. Only recombinant human SOD1 treated with a reducing agent during purification was an efficient Human SOD1 substrate for SOD1 RT-QuIC.

FIG. 10 shows transmission electron microscopy (TEM) images after a SOD1 RT-QuIC experiment. Fibrils propagated from negative control human spinal cord (far left), human SOD1 familial ALS spinal cord (middle), and a human sporadic ALS spinal cord (far right). Four wells of each sample were scraped and imaged. Scale bar=500 nm.

FIG. 11 shows SOD1 seeding activity at 105 spinal cord dilutions from an ALS patient with a SOD1 mutation, a sALS patient, and two human negative controls.

FIG. 12 shows immunodepletion of SOD1 seeding activity using anti-SOD1 antibodies. (A) SOD1 RT-QuIC of supernatants of sALS spinal cord homogenate exposed to beads alone (purple), or beads linked to either a pan-SOD1 antibody (pink) or an isotype-control antibody (gray). (B) Panel shows the use of the C4F6 SOD1 antibody (orange) and a different isotype-control antibody treatment (black). A non-ALS negative control spinal cord homogenate was included without antibody treatment and remained ThT-negative throughout. All samples were done using 5×10⁻³ spinal cord tissue dilutions. ThT fluorescence traces from individual quadruplicate wells are shown.

FIG. 13 shows SOD1 RT-QuIC analysis of cervical spinal cord homogenates from SOD1 familial ALS (A) patient 2 and (B) patient 5 at 10⁻³-10⁻⁵ dilutions. Data from non-ALS control cervical spinal cord (10⁻³ dilution) are shown for comparison.

FIG. 14 shows SOD1 RT-QuIC analysis of sporadic ALS cases using cervical (A, B) or thoracic (C, D) spinal cord homogenates from sporadic ALS patients (A) 5, (B) 3, (C) 6, and (D) 8 at the designated dilutions. Data from non-ALS cervical and thoracic controls (10⁻³ dilution) are shown for comparison.

FIG. 15 shows SOD1 RT-QuIC of cervical spinal cord homogenates from C90RF72 fALS (A) patient 2 and (B) patient 4 compared to non-ALS negative controls 1 and 2 at the designated dilutions.

FIG. 16 shows a plot of 50% ThT fluorescence (RFU) versus lag phase for several ALS patient spinal cord homogenates at 10⁻³ (triangles), 10⁻⁴ (circles), and 10⁻⁵ (rectangles) dilutions. Colors in legend represent each ALS cord type. At equivalent spinal cord dilutions, ALS patients have similar lag phases, with higher 50% ThT fluorescence in SOD1 fALS patient cervical cords and sporadic ALS thoracic cords. Dashed and dotted are polynomial fits to each ALS cord type.

FIG. 17 shows fast protein liquid chromatogram of BL21 E. coli expressed human SOD1 WT eluted via an imidazole gradient. The later eluting peak, peak 2 (pk2, 120-140 mL), is human SOD1 WT (Left), determined via SDS-PAGE (no heat, no reducing agent) stained with Coomassie (Right), showing pk2 is in dimer and monomer equilibrium. Peak 2 was further characterized to confirm mass and identity.

FIG. 18 shows native PAGE immunoblot for SOD1 of postmortem human ALS patient spinal cord homogenates and human negative control spinal cord homogenates. 0.05 μg of purified BL21 SOD1 WT. 1.93 μg of total protein from spinal cord homogenates of two donors each of negative controls (Ctrl), SOD1 fALS, C9ORF72 fALS, and sALS, are indicated above the blot. Apparent molecular weights in kD are indicated on the left- and right-hand sides alongside a molecular weight standard (MW).

FIG. 19 shows ThT fluorescence (RFU) versus time (hours) for five human negative control spinal cord homogenates run in SOD1 RT-QuIC assay. A). 10⁻³ spinal cord dilutions. B). 10⁻⁴ spinal cord dilutions. C). 10⁻⁵ spinal cord dilutions.

FIG. 20 shows representative images of electron micrograph of SOD1 RT-QuIC end products (a) Familial ALS patient with abnormal C90RF72 expansion (b) SOD1 fALS patient 1 (c) Sporadic ALS patient 5 and (d) negative control patient 1. All reactions were seeded with 10⁻⁴ dilution of spinal cord. Scale bar −200 nm.

FIG. 21 shows the kinetics of SOD1 RT-QuIC data from ALS spinal cord homogenates from at 10⁻⁴ dilution. Solid curves are fits of the data to a sigmoidal equation.

FIG. 22 shows human non-mutant SOD1 cDNA and amino acid sequence information (SEQ ID NO:5) and an amino acid sequence (underline is tag) (SEQ ID NO:4).

FIG. 23 shows a plot of ThT fluorescence (RFU) versus time (hours) for cerebrospinal fluid (CSF) from three negative controls and two sporadic Amyotrophic Lateral Sclerosis patients.

FIG. 24 shows a plot of ThT fluorescence (RFU) versus time (hours) for cerebrospinal fluid (CSF) from negative control (2A) and sporadic ALS patient #308.

FIG. 25 shows a plot of ThT fluorescence (RFU) versus time (hours) for cerebrospinal fluid (CSF) from negative control (single donor) and sporadic Amyotrophic Lateral Sclerosis patient #308.

FIG. 26 shows a plot of ThT fluorescence (RFU) versus time (hours) for cerebrospinal fluid (CSF) from negative control (2A) and sporadic Amyotrophic Lateral Sclerosis patient #410.

FIG. 27 shows a plot of ThT fluorescence (RFU) versus time (hours) in SOD1 RT-QuIC assay with cerebrospinal fluid (CSF) from single donor negative control and sporadic Amyotrophic Lateral Sclerosis patient #410.

FIG. 28 shows a plot of ThT fluorescence (RFU) versus time (hours) in SOD1 RT-QuIC assay of postmortem brain tissue (substantia nigra) from patients clinically diagnosed with idiopathic Parkinson's disease or sporadic Alzheimer's disease.

FIG. 29 shows a plot of ThT fluorescence (RFU) versus time (hours) for negative control (a) 6 and (b) 7 cervical spinal cord homogenates run in SOD1 RT-QuIC assay from 10⁻³ to 10⁻⁵ dilution.

FIG. 30 shows immunoprecipitation of sALS patient 4 spinal cord homogenate flow-through after antibody (Ab) capture. Lanes 1 and 7 are molecular weight standards, lane 2 is κ μL of 10% w/v sALS cord input (100%), sALS flow through after Ab capture is shown in lanes 3 (pan-SOD1 Ab, 23%), 4 (pan-SOD1 isotype control, 75%), 5 (C4F6, 21%), and 6 (C4F6 isotype control, 28%). Blue boxes are quantification areas to assess immunodepletion. Percent number is amount remaining SOD1 compared to input. Apparent molecular weights in kDa are indicated on the right-hand side.

FIG. 31 shows a plot of ThT fluorescence (RFU) versus time for cervical spinal cord (CSC) negative control 8, run in the SOD1 RT-QuIC assay from 10⁻³ to 10⁻⁵ dilution.

FIG. 32 shows a plot of ThT fluorescence (RFU) versus time for thoracic spinal cord (TSC) negative control 9 run in SOD1 RT-QuIC assay from 10⁻³ to 10⁻⁵ dilution.

FIG. 33 shows a plot of ThT fluorescence (RFU) versus time for thoracic spinal cord (TSC) negative control 10 run in SOD1 RT-QuIC assay from 10⁻³ to 10⁻⁵ dilution.

FIG. 34 shows a plot of ThT fluorescence (RFU) versus time for cervical spinal cord (CSC) negative control 11 run in SOD1 RT-QuIC assay from 10⁻³ to 10⁻⁵ dilution.

FIG. 35 shows a plot of ThT fluorescence (RFU) versus time for cervical spinal cord (CSC) negative control 12 run in SOD1 RT-QuIC assay from 10⁻³ to 10⁻⁵ dilution.

FIG. 36 shows a plot indicating 50% ThT fluorescence (RFU) versus lag phase (hours) of SOD1 seeding activity in motor cortex from sporadic ALS patients and other non-ALS neurological disease controls.

FIG. 37 shows a plot of ThT fluorescence (RFU) versus time for motor cortex (MCx) negative control 8 run in SOD1 RT-QuIC assay from 10⁻³ to 10⁻⁵ dilution.

FIG. 38 shows a plot of ThT fluorescence (RFU) versus time for motor cortex (MCx) negative control 9 run in SOD1 RT-QuIC assay from 10⁻³ to 10⁻⁵ dilution.

FIG. 39 shows a plot of ThT fluorescence (RFU) versus time for motor cortex (MCx) negative control 9 run in SOD1 RT-QuIC assay from 10⁻³ to 10⁻⁵ dilution.

FIG. 40 shows immunodepletion of SOD1 seeding activity using anti-SOD1 antibodies. (a) SOD1 RT-QuIC of supernatants of sALS spinal cord homogenate exposed to beads alone (green), or beads linked to either a pan-SOD1 antibody (pink) or an isotype-control antibody (gray). (b) Same as panel (a), except for the use of the C4F6 SOD1 antibody (orange) and a different isotype-control antibody treatment (gray). A non-ALS negative control spinal cord homogenate was included without antibody treatment and remained ThT-negative throughout. All samples at 5×10⁻³ spinal cord tissue dilutions. ThT fluorescence traces from individual quadruplicate wells are shown.

FIG. 41 shows a SOD1 RT-QuIC plot of cervical spinal cord homogenates from SOD] familial ALS patients 2 (a) and 5 (b) at 10\hat{ }3-10\hat{ }5 dilutions. Data from non-ALS control cervical spinal cord (10\hat{ }-3 dilution) shown.

FIG. 42 shows a SOD1 RT-QuIC plot of sporadic ALS cases using cervical and thoracic spinal cord homogenates. In (a), sporadic ALS patients 5 (cervical) and 6 (thoracic) are shown, and in (b), sporadic ALS patients 3 (cervical) and 8 (thoracic) are shown at the designated dilutions. Data from non-ALS control spinal cords are shown for comparison.

FIG. 43 shows a SOD1 RT-QuIC plot of cervical spinal cord homogenates from C9ORF72 fALS patients 2 (a) and 4 (b) compared to non-ALS control spinal cord at the designated dilutions.

FIG. 44 shows 50% ThT fluorescence (RFU) versus lag phase (hours) for ALS patient spinal cord homogenates and neurological negative control spinal cords at 10⁻³ (triangles), 10⁻⁴ (circles), and 10⁻⁵ (rectangles) dilutions. Colors represent ALS type. Dashed lines are fits to equation 3 for each ALS type, except sALS cervical cords.

FIG. 45 shows sALS patients 6, 7, 8, 9, 10 motor cortex (a) and thoracic cord (b) versus tissue-matched controls at 10⁻⁴ dilution.

FIG. 46 shows 50% ThT fluorescence (RFU) versus lag phase (hours) for motor cortex dilutions (yellow circles) and thoracic spinal cord dilutions (green circles) at 10⁻⁴ for sALS patients 6, 7, 8, 9, 10 with neurological negative controls 8, 9, 10, 11, 12 (white and blue).

FIG. 47 shows receiver operator characteristic curves for SOD1 RT-QuIC assay at (a) 10\hat{ }-3 spinal cord dilutions (threshold 5,000 RFU and 125 hours) (a) and (b) 10\hat{ }-4 spinal cord dilutions (threshold 5,000 RFU and 175 hours).

FIG. 48 shows SOD1 seeding activity correlates with disease progression in a ALS (SOD1 mutant) animal model. Lumbar spinal cords (LSC) or thoracic spinal cords (TSC) from 11 week or 7 week old mice expressing human SOD1 G93A transgene or no SOD1 G93A transgene were extruded, homogenized, and examined for seeding activity of SOD1 at 10⁻³ dilution. 11 week old mice expressing SOD1 G93A (orange and green) have shortened lag phase and higher ThT fluorescence relative to 7 week old SOD1 G93A mice (blue and pink) and tissue matched negative controls that remained ThT negative throughout experiment. Hemizygous male mice expressing a human SOD1 G93A transgene at 7 weeks and 11 weeks of age were purchased from Jackson Labs (Strain #: 002726, B6SJL-Tg (SOD1*G93A)1Gur/J) with age matched control mice. Animal euthanasia and cervical dislocation were performed. Spinal cords were extruded and sectioned by their lumbar (LSC) or thoracic (TSC) section. Spinal cord sections were homogenized at 10% w/v as described in [0156]. The SOD1 RT-QuIC assay was done as described herein in Paragraph [0148]. Data analysis was done as described herein in Paragraphs [0171]-[0176].

FIG. 49 shows SOD1 seeding activity detected in antemortem cerebrospinal fluid (CSF) of sporadic ALS patients (i.e., not linked to a genetic mutation that causes ALS). Results indicate SOD1 seeding activity is a disease progression biomarker for sporadic ALS. CSF was collected from sporadic ALS patients at different time points during their disease. Table 5 in Example 2 below shows sporadic ALS patient sex, age, and their ALS functional rating scale revised (ALSFRS-R) slope decline, which is a measure of how quickly an ALS patient is progressing with their disease (more negative the slope, the faster the progression). The time CSF was collected in their disease, and SOD1 RT-QuIC kinetic parameters are also shown.

FIG. 50 shows the data in FIG. 49 fit to a sigmoidal model to extract lag phase (hours) and 50% ThT fluorescence (RFU) from each sporadic ALS patient's CSF. A plot of lag phase versus disease duration at CSF collection is shown in this FIG. 50, indicating lag phase correlates with progression of sporadic ALS and ALSFRS-R slope decline (i.e., more negative the faster the progression of disease).

FIG. 51 shows the kinetic curves in FIG. 49 were fit to sigmoidal model to extract 50% ThT fluorescence (RFU) and lag phase (hours). Mean 50% ThT (RFU) vs. lag phase (hours) was plotted and linear regression analysis performed to determine negative correlation between these two parameters. The linear correlation coefficient R=0.99.

FIG. 52 shows SOD1 seeding activity in brain regions of three Alzheimer's patients (hippocampus, frontal cortex, motor cortex) at 10⁻³ dilution. The SOD1 RT-QuIC assay was done as described herein in Paragraph [0148]. Data analysis was done as described herein in Paragraphs [0171]-[0176].

FIG. 53 shows SOD1 seeding activity in brain regions of three Alzheimer's patients (hippocampus, frontal cortex, motor cortex) versus controls at 10⁻⁴ dilution. Data analysis was done as described herein in Paragraphs [0171]-[0176].

FIG. 54 shows SOD1 seeding activity in brain regions of three Alzheimer's patients (hippocampus, frontal cortex, motor cortex) versus controls at 10⁻⁵ dilution. Data analysis was done as described herein in Paragraphs [0171]-[0176].

FIG. 55 shows analysis of free cysteine residues in SOD1 indicating what cysteine residues have disulfide bond in SOD1 for RT-QuIC assay as described in Example 3. (A) β-mercaptoethanol (βME) treated SOD1 was incubated with 15 mM iodoacetamide (IOA) prior to protein mass determination by ESI-MS. This experiment labels free thiols with carboxymethylation, but not cysteines involved in disulfide bonds IOA adds a ±57 mass signature to free cysteine (Cys) residues. Deconvoluted protein mass spectra show the predominant mass of 17,980 Da, which is indicative of 2 labeled Cys residues. (B) Untreated (no βME) SOD1 incubated with IOA results in all four Cysteine residues being labeled, indicative of no disulfide bonding. Cys75-Cys164 had little to no labeling of IOA indicating these residues are the intramolecular disulfide bond in the SOD1 substrate for RT-QuIC.

### DETAILED DESCRIPTION

In one embodiment, the present disclosure relates methods of preparing a purified human Superoxide Dismutase 1 (SOD1) substrate for use in a real-time quaking induced conversion (RT-QuIC) or seed amplification assay (SAA).

In a second embodiment, the present disclosure relates to methods for detecting the presence of at least one misfolded form of human Superoxide Dismutase 1 (SOD1) in a biological sample obtained from a human subject, suspected of having or having at least one neurodegenerative disease (e.g., ALS, Parkinson's disease or Alzheimer's disease), using an improved real-time quaking induced conversion (RT-QuIC) assay. In some aspects, the methods employ the purified human SOD1 substrate described in the first embodiment as well as a unique buffer and reaction conditions (e.g., pH, shaker speed, and temperature) to identify misfolded forms of human SOD1 in various different types of ALS.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and”, and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 69, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

In instances where a range of values is provided, it is understood that every value within that range, to the nearest tenth of the unit of the lower limit unless otherwise indicated by the context, including any other stated or intervening values within that range, falls within the scope of the present disclosure. Both the upper and lower limits of these smaller ranges may be included independently within the smaller ranges and are also considered part of the present disclosure, except where explicitly excluded by stated limits within the range. If the stated range includes one or both of its limits, ranges excluding either or both of these included limits are also considered part of the present disclosure.

“Bead” and “particle” are used herein interchangeably and refer to a substantially spherical solid support. One example of a bead or particle is a microparticle. Microparticles that can be used herein can be any type known in the art. For example, the bead or particle can be a magnetically susceptible (or responsive) bead or particle (See, for example, U.S. Pat. Nos. 4,230,685 4,554,088 and 4,628,037, all of which are herein incorporated by reference) or magnetic particle, as used interchangeable herein. Another example of a bead or particle is a magnetic or magnetically susceptible beads or particles.

“Neurodegenerative disease” as used herein refers to the progressive degeneration of neurons in, e.g., the central nervous system (CNS), characterized by molecular and genetic changes in nerve cells that result in nerve cell degeneration and ultimately nerve dysfunction and death. Examples of neurodegenerative diseases include, but are not limited to, tauopathies, Alzheimer's disease (AD), amyotrophic lateral sclerosis (ALS), Huntington's disease (HD), Parkinson's disease (PD), prion disease, vascular dementia, progressive supranuclear palsy (PSP) and corticobasal degeneration (CBD).

“Rapid buffer exchange” or “rapid online buffer exchange” as used interchangeably herein refers to separation of proteins and non-volatile small molecules using a short size-exclusion column or filter which removes salts and contaminants less than about 10 kDa, and in some aspects, less than 8 kDa. Rapid buffer exchange or rapid online buffer exchange has been described in Zachary L. VanAernum, et al., Nat. Protoc., 15 (3): 1132-1157 (March 2020), the contents of which are herein incorporated by reference.

“Real-time quaking induced conversion” (RT-QuIC) technology, seed amplification assay (SAA) or assay as used herein refers to in vitro amplification technology for detection of the abnormal form of prion protein (PrP^{Sc}) in biological samples, such as cerebrospinal fluid (CSF), described in Ryuichiro Atarashi et al., “Real-time quaking-induced conversion: A highly sensitive assay for prion detection,” Prion, 5:3, 150-153 (July/August/September 2011), the contents of which are herein incorporated by reference.

A “reducing agent” as used herein, refers to a substance that can be used to break disulfide bonds within and between proteins. Examples of reducing agents that can be used in the present invention, include, but are not limited to, β-mercaptoethanol (βME), Dithiothreitol (DTT), tris(2-carboxyethyl)phosphine (TCEP), or any combinations thereof.

“Sample,” “test sample,” “specimen,” “sample from a subject,” “biological sample,” and “patient sample” as used interchangeably herein may be a sample of cerebrospinal fluid, tissue, whole blood, serum, plasma, saliva, nasal brushings, skin, urine, tears, or any combinations thereof. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

In some aspects, the sample is cerebrospinal fluid. In some aspects, the sample is tissue. In some aspects, the sample is spinal cord tissue (either a sample from a living patient or a sample from a cadaver). In other aspects, the sample is brain tissue. In still other aspects, the biological sample is whole blood. In still further aspects, the sample is serum. In still further aspects, the sample is plasma. In still other aspects, the sample is saliva. In still yet other aspects, the sample is nasal brushings. In still yet other aspects, the sample is urine. In still other aspects, the sample is skin. In still other aspects, the sample is tears.

“Solid phase” or “solid support” as used interchangeably herein, refers to any material that can be used to attach and/or attract and immobilize one or more proteins (e.g., such as one or more misfolded proteins). The solid phase can be chosen for its intrinsic ability to attract and immobilize one or more proteins. Alternatively, the solid phase can have affixed thereto a linking agent that has the ability to attract and immobilize the one or more proteins. For example, the linking agent can include a charged substance that is oppositely charged with respect to the protein itself. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art. In some aspects, the solid support can be a magnetically susceptible bead or particle. In another aspect, the solid support can be a 96-well plate or 384-well plate.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. In some embodiments, the subject is a human. A “subject in need” of treatment for a particular condition, e.g. a neurodegenerative condition, can be a subject suspected of having that condition, diagnosed as having that condition, already treated or being treated for that condition, not treated for that condition, or at risk of developing that condition.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods of Preparing a Purified Human Superoxide Dismutase 1 (SOD1) Substrate for Use in Real-Time Quaking Induced Conversion Assay

In one embodiment, the present discloses relates to methods of preparing a human Superoxide Dismutase 1 (SOD1) substrate for use in a real-time quaking induced conversion (RT-QuIC) assay. In one aspect, the method involves obtaining or providing a sample of cells that express at least one human SOD1 protein. The cells expressing the at least one human SOD1 protein are not critical and can be any type of cells known in the art in the art useful for expressing proteins such as, for example, E. coli cells, Chinese Hamster Ovary (CHO) cells, Human embryonic kidney (HEK) cells, Hela cells, baby hamster kidney (BHK21) cells, insect cells, murine myeloma cells, etc.

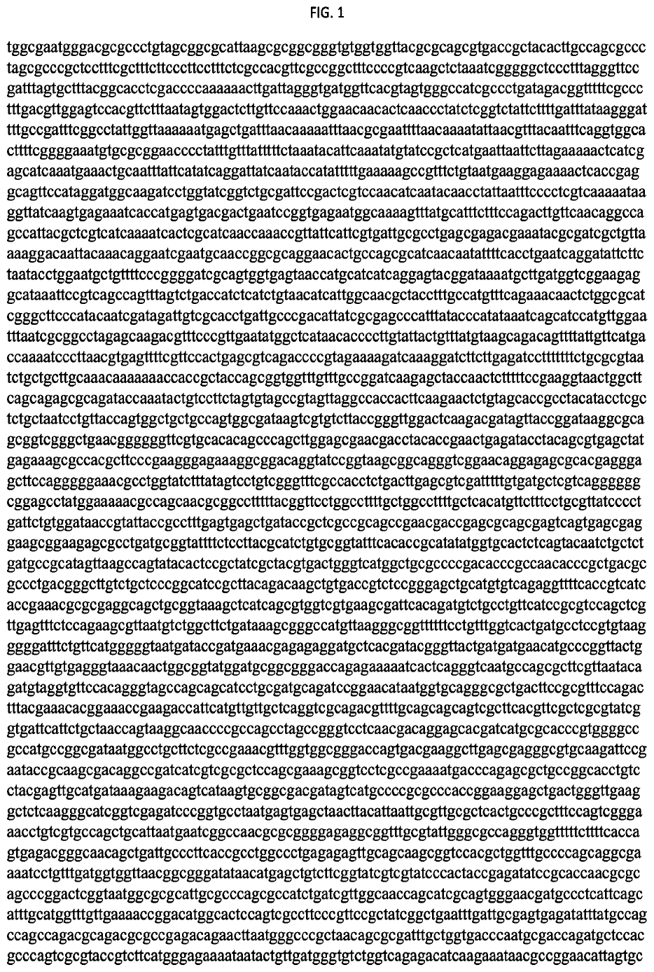

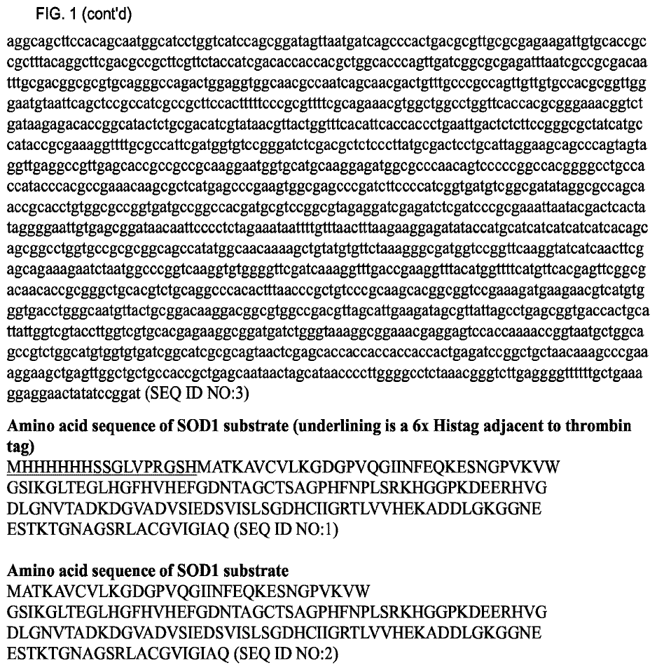

In some aspects, the at least one human SOD1 protein expressed by the cells has an amino acid sequence of SEQ ID NO:1 or SEQ ID NO:2. In other aspects, the at least one human SOD1 protein expressed by the cells has an amino acid sequence having at least 80%, at least 85%, at least 90%, at least 91%, at least 92%, at least 93%, at least 94%, at least 95%, at least 96%, at least 97%, at least 98%, at least 99%, or at least 100% identity to SEQ ID NO:1 or SEQ ID NO:2. For example, in some aspects, a cDNA sequence of human SOD1, such as SEQ ID NO:3 (shown in FIG. 1), can be transfected into a suitable vector known in the art (e.g., such as a pET28(+) vector using routine techniques known in the art. Once the vector is prepared, it can be transformed into E. coli cells (such as BL21 E. coli competent cells) using routine techniques known in the art to produce cells that express the human SOD1 protein which can be used in the methods described herein.

Once a sample of cells expressing the human SOD1 protein is obtained or provided, chromatography is performed on the sample to produce an eluate, using routine techniques known in the art. The type of chromatography performed is not critical and can be any type of column chromatography such as affinity chromatography (e.g., Protein A chromatography), liquid chromatography (e.g., including high performance liquid chromatography), ion exchange chromatography, size exclusion chromatography, or any combinations thereof.

Once the eluate is produced it can be further processed in one of two ways. The first way in which the eluate can be further processed is by incubating the eluate for a period of time of at least about 6 hours and at a temperature of about 20° C. to about 25° C. in at least 20 mM to about 30 mM of at least one buffer having a pH between about 6.0 to about 8.0 to produce a purified human SOD1 substrate. More specifically, in some aspects, the eluate is incubated for at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours to produce a purified human SOD1 substrate.

In other aspects, the temperature of the incubation during this first processing is about 20° C. to about 22° C. In some aspects, the eluate is incubated at a temperature of about 20° C., about 21° C., about 22° C., about 23° C., about 24° C., or about 25° C.

In still yet further aspects, the buffer used during this first processing is any buffer that has a pH between about 6.0 and about 8.0. More specifically, the buffer can have a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. Examples of buffers that can be used for this further processing include, for example, a Tris (trisaminomethane) buffer, a sodium phosphate buffer, HEPES (N-2-hydroxyethylpiperazine-N′-2-ethanesulfonic acid), Tris-HCl, or any combinations thereof.

In some aspects, the concentration of the buffer that can be used in the first processing is between about 10 mM to about 90 mM. In further aspects, the concentration of the buffer used in the first processing is between about 10 mM to about 50 mM, about 10 mM to about 30 mM, about 10 mM to about 25 nM. In yet further aspects, the concentration of the buffer is about 25 mM. In yet even further embodiments, the buffer is a Tris buffer, the concentration of which is about 25 mM.

After this further processing, the eluate can be subjected to yet further (e.g., additional) processing steps. For example, in some aspects, after the incubation, the eluate can be subject to centrifugation, using routine techniques in the art. The supernatant produced from such centrifugation can then be filtered to produce a purified human SOD1 substrate. This purified human SOD1 substrate can be used in the methods described in Section 3, below.

The second way the eluate can be processed is by using rapid online buffer exchange, exchange using an exclusion column or filter which removes any salts and/or contaminants having a molecular weight less than about 10 kDa or performing dialysis using routine techniques known in the art. The rapid online buffer exchange, exchange using an exclusion column or dialysis used in this second processing be performed in the presence of a buffer having a pH between about 6.0 to about 8.0 for at least 10 minutes, at least 15 minutes, at least 20 minutes, at least 30 minutes, at least 45 minutes, at least one 1 hours, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

The pH of the buffer used in the second processing is between 6.0 to about 8.0. More specifically, the buffer can have a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. Examples of buffers that can be used for this second processing include, for example, a Tris (trisaminomethane) buffer, a sodium phosphate buffer, HEPES (N-2-hydroxyethylpiperazine-N′-2-ethanesulfonic acid), Tris-HCl, or any combinations thereof. In some aspects, the concentration of the buffer that can be used in the second processing is between about 10 mM to about 90 mM. In further aspects, the concentration of the buffer used in the second processing is between about 10 mg to about 50 mM, about 10 mg to about 30 mM, about 10 mM to about 25 nM. In yet further aspects, the concentration of the buffer is about 25 mM. In yet even further embodiments, the buffer is a Tris buffer, the concentration of which is about 25 mM.

After this further processing, the eluate can be subjected to yet further (e.g., additional) processing steps. For example, in some aspects, after the rapid online buffer exchange, exchange using an exclusion column or filter or dialysis, the eluate can be subject to centrifugation, using routine techniques in the art. The supernatant produced from such centrifugation can then be filtered to produce a purified human SOD1 substrate. This purified human SOD1 substrate can be used in the methods described in Section 3, below.

### 3. Methods for Detecting the Presence of at Least One Misfolded Form of Human Superoxide Dismutase 1 (SOD1) in a Biological Sample Obtained from a Human Subject

In a second embodiment, the present disclosure relates to methods for detecting the presence of at least one misfolded form of human Superoxide Dismutase 1 (SOD1) in a biological sample obtained from a human subject, suspected of having or having at least one neurodegenerative disease (e.g., ALS, Parkinson's disease or Alzheimer's disease), using an improved real-time quaking induced conversion (RT-QuIC) assay. In some aspects, the methods employ the purified human SOD1 substrate described in the first embodiment as well as a unique buffer and reaction conditions (e.g., pH, shaker speed, and temperature) to identify misfolded forms of human SOD1 in different types of ALS.

In one aspect, the above method involves performing a RT-QuIC assay on a biological sample obtained from a human subject. In some aspects of the above method, the human subject is suspect of having a neurodegenerative disease, such as, for example, ALS, Parkinson's disease or Alzheimer's disease. In other aspects, the subject has been previously diagnosed as having a neurodegenerative disease, such as, for example, ALS, Parkinson's disease, or Alzheimer's disease. Once a biological sample is obtained or produced from a human subject, RT-QuIC is performed using (a) the purified human SOD1 substrate produced as described in Section 2; and (b) under a unique set of reaction conditions. These reaction conditions include performing the RT-QuIC assay: (1) using a reaction mixture that comprises at least one buffer which comprises: (a) about 0.5 M to about 0.75 M guanidine HCl or urea; and (b) about 0.01 M to about 0.05 M sodium acetate; (2) at a pH of about 3.5 to about 4.5; (3) using a shaker speed of about 300 rpm to about 700 rpm; and (4) a temperature of about 35° C. to about 42° C. In addition, at least one fluorescent compound is used in the RT-QuIC assay.

After the assay is performed or conducted, the presence or absence of a detectable signal from at least one fluorescent compound is detected using routine techniques known in the art. The presence of a detectable signal in the biological sample indicates the presence of at least one misfolded form of SOD1 in the biological sample. The absence of at least one detectable signal in the biological sample indicates the absence of at least one misfolded form of SOD1 in the biological sample. Optionally, in some aspects, the assay can be performed using one or more solid supports (e.g., beads or a 96-well plate or a 384-well plate), to which the misfolded form of SOD1 is bound.

In some aspects of the above method, the purified human SOD1 substrate produced as described in Section 2, can be used in the RT-QuIC assay in a concentration of from about 10 μM to about 60 μM. In some aspects, the concentration of the purified human SOD1 substrate used in RT-QuIC assay is about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 31 μM, about 32 μM, about 33 μM, about 34 μM, about 35 μM, about 36 μM, about 37 μM, about 38 μM, about 39 μM, about 40 μM, about 41 μM, about 42 μM, about 43 μM, about 44 μM, about 45 μM, about 46 μM, about 47 μM, about 48 μM, about 49 μM, about 50μ, about 51μ, about 52μ, about 53μ, about 54μ, about 55μ, about 56μ, about 57 μM, about 58 μM, about 59 μM, or about 60 μM. In some aspects, the concentration of the purified human SOD1 substrate used in RT-QuIC assay is about 30 μM. The concentration of purified human SOD1 substrate in the RT-QuIC can be confirmed using routine techniques known in the art, such as, for example, by use of a bicinchoninic acid (BCA) assay or measuring absorbance at 280 nm using extinction coefficient from 5,350 to 5,750 M⁻¹ cm⁻¹.

In some aspects of the above method, the reaction buffer comprises from (a) about 0.5 M, about 0.6 M, about 0.7M, or about 0.75 M guanidine HCl or about 0.5 M, about 0.6 M, about 0.7M, or about 0.75 M urea; and (b) about 0.01 M, about 0.02 M, about 0.03 M, about 0.04M, or about 0.05 M sodium acetate. In other aspects of the above method, the reaction buffer can further comprise ethylenediaminetetraacetic acid (EDTA), one or more reducing agents, one or more salts (e.g., sodium chloride, potassium chloride, calcium chloride, etc.), or any combinations thereof. In other aspects of the above method, the reaction buffer comprises EDTA, one or more reducing agents and one or more salts. In still yet further aspects of the above method, the reaction buffer comprises EDTA, β-mercaptoethanol, and sodium chloride.

In further aspects of the above method, the RT-QuIC assay is performed at a pH of about 3.9 to about 4.0 or about 3.8 to about 4.1. In yet other aspects, the pH of about 3.5, about 3.6, about 3.7, about 3.8, about 3.9, about 4.0, about 4.1, about 4.2, about 4.3, about 4.4, or about 4.5.

In still yet further aspects of the above method, the RT-QuIC assay is performed of using a shaker speed of about 300 rpm, about 310 rpm, about 320 rpm, about 330 rpm, about 340 rpm, about 350 rpm, about 360 rpm, about 370 rpm, about 380 rpm, about 390 rpm, about 400 rpm, about 400 rpm, about 410 rpm, about 420 rpm, about 430 rpm, about 440 rpm, about 450 rpm, about 460 rpm, about 470 rpm, about 480 rpm, about 490 rpm, about 500 rpm, about 510 rpm, about 520 rpm, about 530 rpm, about 540 rpm, about 550 rpm, about 560 rpm, about 570 rpm, about 580 rpm, about 590 rpm, about 600 rpm, about 610 rpm, about 620 rpm, about 630 rpm, about 640 rpm, about 650 rpm, about 660 rpm, about 670 rpm, about 680 rpm, about 690 rpm, or about 700 rpm.

In still further aspects of the above method, the RT-QuIC assay is performed at a temperature of about 35° C., about 36° C., about 37° C., about 38° C., about 39° C., about 40° C., about 41° C., or about 42° C.

Moreover, any fluorescent compound can be used in the RT-QuIC assay. Examples of fluorescent compounds that can be used are one or more amyloid binding dyes. Examples of amyloid binding dyes include, but are not limited to, congo red, iodine-sulfuric acid, Thioflavin T or Thioflavin S, crystal violet, methyl violet, BTA-1, chrysamine G, ANS (1-anilinonaphthalene-8-sulfonic acid), bisANS (4,4′-dianilino-1,1′-binaphthyl-5,5′-disulfonic acid), Nile red, K114 ((trans, trans)-1-bromo-2,5-bis(4-hydroxystyryl)benzene), FSB, curcumin, nanocurcumin, In some aspects, the concentration of at least one fluorescent compound used in the RT-QuIC assay is from about 5 μM to about 100 μM, about 5 μM to about 75 μM, about 5 μM to about 50 μM, about 5 μM to about 30 μM, or about 5 μM to about 20 μM. In some aspects, the fluorescent compound is Thioflavin T which is used in a concentration of about 20 μM.

If at least one misfolded form of SOD1 is detected in the biological sample, the subject can be diagnosed as having a neurodegenerative disease. For example, in some aspects, the subject may be diagnosed as having motor neuron disease. In some aspects, the motor neuron disease is ALS. In yet further aspects, the subject may be diagnosed as having familial ALS or sporadic ALS. In yet further aspects, the above method may also be useful in detecting different conformers of misfolded SOD1 such as in patients suffering from familial or sporadic ALS. In yet other aspects, the subject may be diagnosed as having Parkinson's disease. In still yet other aspects, the subject may be diagnosed as having Alzheimer's.

Once the subject has been diagnosed as having a neurodegenerative disease, the methods described herein can be repeated on an as needed basis by a clinician to monitor the status of the patient to see if the neurodegenerative disease is progressing and/or responding to treatment with a therapeutic agent. Treatments for neurodegenerative diseases include: (a) administration of one or more pharmacological agents (e.g., cholinesterase inhibitors (e.g., donepezil, rivastigmine), antisense oligonucleotides (e.g. FDA approved tofersen or Qalsody), copper or zinc compounds that could stabilize misfolded SOD1, antibodies, such as ones specific to SOD1, NMDA receptor antagonists (e.g., memantine), and their combinations, dopamine receptor agonists (e.g., apomorphine), dopamine precursors (e.g., levodopa), monamine oxidase inhibitors (MAO-B), or any combinations thereof); (b) cognitive therapy (e.g., exercises aimed at training memory, attention, and thinking); (c) physical exercises (e.g., aerobic, strength, or combination thereof); (d) speech and swallowing therapy; (e) ergotherapy; (f) brain stimulation; or (g) any combinations of (a)-(f).

The present disclosure has multiple aspects, illustrated by the following non-limiting examples.

### EXAMPLES

### Example 1: Preparation of Human SOD1 Plasmid

cDNA encoding full length non-mutant (wild-type) human SOD1 was manufactured by GenScript (Piscataway NJ, USA) and placed in the pET 28a(+) vector with an N-terminus 6×His-tag. The human thrombin sequence containing a glycine, serine, serine sequence was removed to prevent any possible cleavage of the 6×His-tag during protein expression and purification. The cDNA sequence for SOD1 was confirmed by GenScript, and after expression and purification of SOD1, its mass was confirmed by electrospray ionization liquid chromatography mass spectrometry (LC-MS).

cDNA sequence of SOD1 substrate:
(SEQ ID NO: 3)
tggcgaatgggacgcgccctgtagcggcgcattaagcgcggcgggtgtgg
tggttacgcgcagcgtgaccgctacacttgccagcgccctagcgcccgct
cctttcgctttcttcccttcctttctcgccacgttcgccggctttccccg
tcaagctctaaatcgggggctccctttagggttccgatttagtgctttac
ggcacctcgaccccaaaaaacttgattagggtgatggttcacgtagtggg
ccatcgccctgatagacggtttttcgccctttgacgttggagtccacgtt
ctttaatagtggactcttgttccaaactggaacaacactcaaccctatct
cggtctattcttttgatttataagggattttgccgatttcggcctattgg
ttaaaaaatgagctgatttaacaaaaatttaacgcgaattttaacaaaat
attaacgtttacaatttcaggtggcacttttcggggaaatgtgcgcggaa
cccctatttgtttatttttctaaatacattcaaatatgtatccgctcatg
aattaattcttagaaaaactcatcgagcatcaaatgaaactgcaatttat
tcatatcaggattatcaataccatatttttgaaaaagccgtttctgtaat
gaaggagaaaactcaccgaggcagttccataggatggcaagatcctggta
tcggtctgcgattccgactcgtccaacatcaatacaacctattaatttcc
cctcgtcaaaaataaggttatcaagtgagaaatcaccatgagtgacgact
gaatccggtgagaatggcaaaagtttatgcatttctttccagacttgttc
aacaggccagccattacgctcgtcatcaaaatcactcgcatcaaccaaac
cgttattcattcgtgattgcgcctgagcgagacgaaatacgcgatcgctg
ttaaaaggacaattacaaacaggaatcgaatgcaaccggcgcaggaacac
tgccagcgcatcaacaatattttcacctgaatcaggatattcttctaata
cctggaatgctgttttcccggggatcgcagtggtgagtaaccatgcatca
tcaggagtacggataaaatgcttgatggtcggaagaggcataaattccgt
cagccagtttagtctgaccatctcatctgtaacatcattggcaacgctac
ctttgccatgtttcagaaacaactctggcgcatcgggcttcccatacaat
cgatagattgtcgcacctgattgcccgacattatcgcgagcccatttata
cccatataaatcagcatccatgttggaatttaatcgcggcctagagcaag
acgtttcccgttgaatatggctcataacaccccttgtattactgtttatg
taagcagacagttttattgttcatgaccaaaatcccttaacgtgagtttt
cgttccactgagcgtcagaccccgtagaaaagatcaaaggatcttcttga
gatcctttttttctgcgcgtaatctgctgcttgcaaacaaaaaaaccacc
gctaccagcggtggtttgtttgccggatcaagagctaccaactctttttc
cgaaggtaactggcttcagcagagcgcagataccaaatactgtccttcta
gtgtagccgtagttaggccaccacttcaagaactctgtagcaccgcctac
atacctcgctctgctaatcctgttaccagtggctgctgccagtggcgata
agtcgtgtcttaccgggttggactcaagacgatagttaccggataaggcg
cagcggtcgggctgaacggggggttcgtgcacacagcccagcttggagcg
aacgacctacaccgaactgagatacctacagcgtgagctatgagaaagcg
ccacgcttcccgaagggagaaaggcggacaggtatccggtaagcggcagg
gtcggaacaggagagcgcacgagggagcttccagggggaaacgcctggta
tctttatagtcctgtcgggtttcgccacctctgacttgagcgtcgatttt
tgtgatgctcgtcaggggggcggagcctatggaaaaacgccagcaacgcg
gcctttttacggttcctggccttttgctggccttttgctcacatgttctt
tcctgcgttatcccctgattctgtggataaccgtattaccgcctttgagt
gagctgataccgctcgccgcagccgaacgaccgagcgcagcgagtcagtg
agcgaggaagcggaagagcgcctgatgcggtattttctccttacgcatct
gtgcggtatttcacaccgcatatatggtgcactctcagtacaatctgctc
tgatgccgcatagttaagccagtatacactccgctatcgctacgtgactg
ggtcatggctgcgccccgacacccgccaacacccgctgacgcgccctgac
gggcttgtctgctcccggcatccgcttacagacaagctgtgaccgtctcc
gggagctgcatgtgtcagaggttttcaccgtcatcaccgaaacgcgcgag
gcagctgcggtaaagctcatcagcgtggtcgtgaagcgattcacagatgt
ctgcctgttcatccgcgtccagctcgttgagtttctccagaagcgttaat
gtctggcttctgataaagcgggccatgttaagggcggttttttcctgttt
ggtcactgatgcctccgtgtaagggggatttctgttcatgggggtaatga
taccgatgaaacgagagaggatgctcacgatacgggttactgatgatgaa
catgcccggttactggaacgttgtgagggtaaacaactggcggtatggat
gcggcgggaccagagaaaaatcactcagggtcaatgccagcgcttcgtta
atacagatgtaggtgttccacagggtagccagcagcatcctgcgatgcag
atccggaacataatggtgcagggcgctgacttccgcgtttccagacttta
cgaaacacggaaaccgaagaccattcatgttgttgctcaggtcgcagacg
ttttgcagcagcagtcgcttcacgttcgctcgcgtatcggtgattcattc
tgctaaccagtaaggcaaccccgccagcctagccgggtcctcaacgacag
gagcacgatcatgcgcacccgtggggccgccatgccggcgataatggcct
gcttctcgccgaaacgtttggtgggggaccagtgacgaaggcttgagcga
gggcgtgcaagattccgaataccgcaagcgacaggccgatcatcgtcgcg
ctccagcgaaagcggtcctcgccgaaaatgacccagagcgctgccggcac
ctgtcctacgagttgcatgataaagaagacagtcataagtgcggcgacga
tagtcatgccccgcgcccaccggaaggagctgactgggttgaaggctctc
aagggcatcggtcgagatcccggtgcctaatgagtgagctaacttacatt
aattgcgttgcgctcactgcccgctttccagtcgggaaacctgtcgtgcc
agctgcattaatgaatcggccaacgcgcggggagaggcggtttgcgtatt
gggcgccagggtggtttttcttttcaccagtgagacgggcaacagctgat
tgcccttcaccgcctggccctgagagagttgcagcaagcggtccacgctg
gtttgccccagcaggcgaaaatcctgtttgatggtggttaacggcgggat
ataacatgagctgtcttcggtatcgtcgtatcccactaccgagatatccg
caccaacgcgcagcccggactcggtaatggcgcgcattgcgcccagcgcc
atctgatcgttggcaaccagcatcgcagtgggaacgatgccctcattcag
catttgcatggtttgttgaaaaccggacatggcactccagtcgccttccc
gttccgctatcggctgaatttgattgcgagtgagatatttatgccagcca
gccagacgcagacgcgccgagacagaacttaatgggcccgctaacagcgc
gatttgctggtgacccaatgcgaccagatgctccacgcccagtcgcgtac
cgtcttcatgggagaaaataatactgttgatgggtgtctggtcagagaca
tcaagaaataacgccggaacattagtgcaggcagcttccacagcaatggc
atcctggtcatccagcggatagttaatgatcagcccactgacgcgttgcg
cgagaagattgtgcaccgccgctttacaggcttcgacgccgcttcgttct
accatcgacaccaccacgctggcacccagttgatcggcgcgagatttaat
cgccgcgacaatttgcgacggcgcgtgcagggccagactggaggtggcaa
cgccaatcagcaacgactgtttgcccgccagttgttgtgccacgcggttg
ggaatgtaattcagctccgccatcgccgcttccactttttcccgcgtttt
cgcagaaacgtggctggcctggttcaccacgcgggaaacggtctgataag
agacaccggcatactctgcgacatcgtataacgttactggtttcacattc
accaccctgaattgactctcttccgggcgctatcatgccataccgcgaaa
ggttttgcgccattcgatggtgtccgggatctcgacgctctcccttatgc
gactcctgcattaggaagcagcccagtagtaggttgaggccgttgagcac
cgccgccgcaaggaatggtgcatgcaaggagatggcgcccaacagtcccc
cggccacggggcctgccaccatacccacgccgaaacaagcgctcatgagc
ccgaagtggcgagcccgatcttccccatcggtgatgtcggcgatataggc
gccagcaaccgcacctgtggcgccggtgatgccggccacgatgcgtccgg
cgtagaggatcgagatctcgatcccgcgaaattaatacgactcactatag
gggaattgtgagcggataacaattcccctctagaaataattttgtttaac
tttaagaaggagatataccatgcatcatcatcatcatcacagcagcggcc
tggtgccgcgcggcagccatatggcaacaaaagctgtatgtgttctaaag
ggcgatggtccggttcaaggtatcatcaacttcgagcagaaagaatctaa
tggcccggtcaaggtgtggggttcgatcaaaggtttgaccgaaggtttac
atggttttcatgttcacgagttcggcgacaacaccgcgggctgcacgtct
gcaggcccacactttaacccgctgtcccgcaagcacggcggtccgaaaga
tgaagaacgtcatgtgggtgacctgggcaatgttactgcggacaaggacg
gcgtggccgacgttagcattgaagatagcgttattagcctgagcggtgac
cactgcattattggtcgtaccttggtcgtgcacgagaaggcggatgatct
gggtaaaggcggaaacgaggagtccaccaaaaccggtaatgctggcagcc
gtctggcatgtggtgtgatcggcatcgcgcagtaactcgagcaccaccac
caccaccactgagatccggctgctaacaaagcccgaaaggaagctgagtt
ggctgctgccaccgctgagcaataactagcataaccccttggggcctcta
aacgggtcttgaggggttttttgctgaaaggaggaactatatccggat
Amino acid sequence of Human SOD1 substrate
(underlining is tag):
(SEQ ID NO: 1)
MHHHHHHSSGLVPRGSHMATKAVCVLKGDGPVQGIINFEQKESNGPVKV
WGSIKGLTEGLHGFHVHEFGDNTAGCTSAGPHFNPLSRKHGGPKDEERHV
GDLGNVTADKDGVADVSIEDSVISLSGDHCIIGRTLVVHEKADDLGKGGN
EESTKTGNAGSRLACGVIGIAQ

### SOD1 Protein Expression and Purification

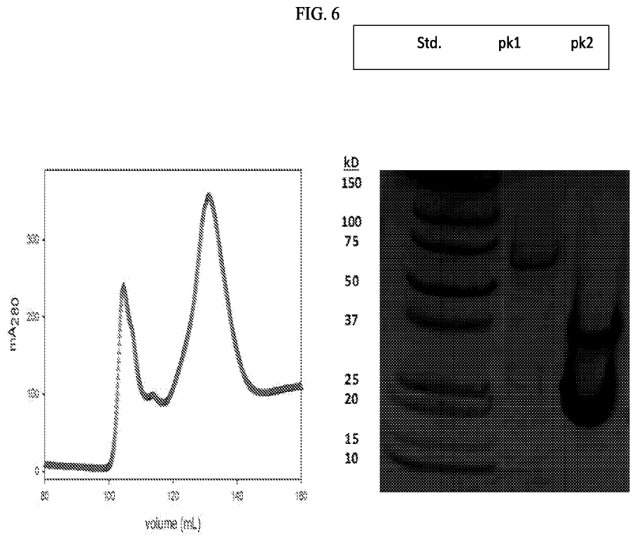

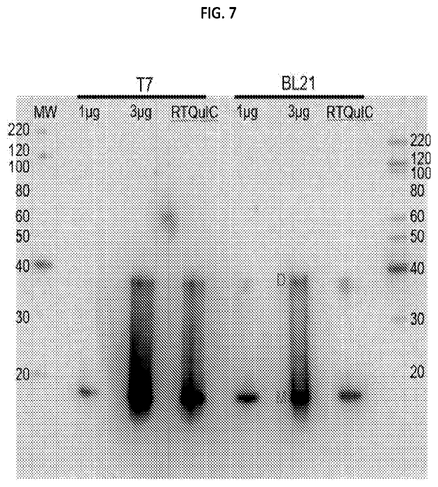

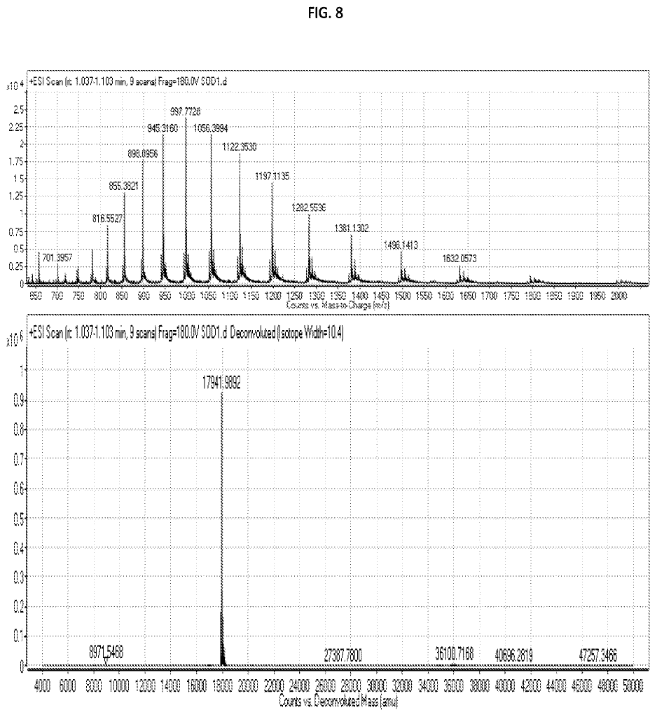

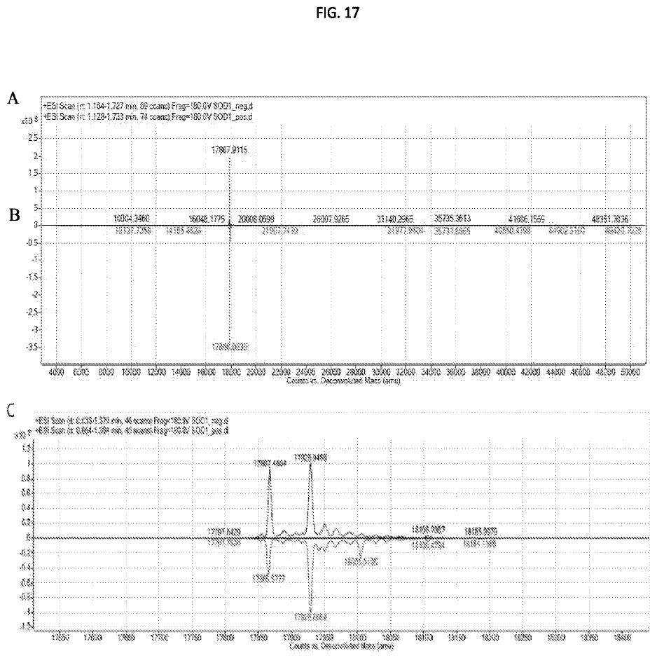

The cDNA encoding human SOD1 was expressed from the pET 28(+) vector by transformation into BL21 (New England Biolabs, Ipswich MA, USA; DE3, #C2727I) E. coli competent cells using the manufacturer's protocol. One liter of sterile 2×YT media was inoculated with 10 mL of suspended transformed E. coli cells and 1 mL of 50 mg/mL kanamycin and placed in a 2.8 L Fernbach flask. Media was placed in an orbital shaker at 250 rpm, 37° C., until OD600 was between 0.6-0.8. At this optical density range, E. coli cells were induced with 1 mM final IPTG concentration overnight (~15 hours) at 30° C. and 150 rpm. The next day, cells were spun for 20 minutes at 7,000 rpm and 4° C. Cell pellets were used immediately or frozen in −20° C. Cells were lysed at a ratio of 40 mL of 0.5 M NaCl, 0.05 M Tris, pH 8.0 (lysis buffer) to 5 g cells and put on ice with 0.5 mg/mL lysozyme and a stir bar. After mixing on ice, cells were sonicated on ice for 5 minutes (30 seconds on/30 seconds off) using a probe sonicator (Sonics VibraCell, Newtown CT, USA). Cell lysate was spun for 20 minutes at 12,000 rpm and 4° C. Supernatant was separated carefully from cell pellet using cheesecloth then poured into a 50 mL Falcon tube for affinity chromatography. Briefly, a Bio-Rad (Hercules CA, USA) NGC fast protein liquid chromatography system with a 5 mL HisTrap HP column (Cytiva, Marlborough MA, USA) was washed with 0.1 M phosphate, 0.3 M NaCl, 0.02 M imidazole, 2 mM β-ME, pH 7.4 buffer (buffer A), and then 50 mL of cell supernatant was applied to the 5 mL HisTrap HP column, washed with buffer A, then eluted with buffer A in a 0.3 M imidazole (0-100%) gradient (buffer B). Observed were two distinct peaks during the imidazole gradient and discovered the later eluting peak with higher imidazole concentration (~50% buffer B) was SOD1 (FIG. 6). Eluted SOD1 was exchanged overnight at room temperature in 25 mM Tris pH 8.0 using 10,000 MWCO Snakeskin dialysis tubing. The next day, dialysate was spun down for 30 min, 15,000 rpm at 4° C. Supernatant was filtered carefully through cheesecloth then concentrated in a 10,000 MWCO Amicon centrifugal concentrator (Millipore Sigma, Burlington MA, USA). Protein purity and identity was assessed with a non-reducing, no heat SDS-PAGE stained with Coomassie (FIG. 6), western blotting (FIG. 7), and electrospray ionization LC-MS (FIG. 17), with expected monomeric SOD1 mass of 17,867.86 Da. Protein concentration was determined using a BCA assay and absorbance at 280 nm with a theoretical extinction coefficient of 5,750 M-1 cm-1 (oxidized) or 5,500 M-1 cm-1 (reduced). The SOD1 concentration via BCA assay and the SOD1 theoretical extinction coefficient from ExPasy were slightly different, with the BCA method producing an experimental extinction coefficient of 5,361.5 M-1 cm-1. Thw5,500 M-1 cm-1 to calculate SOD1 concentration for all experiments.

### Electrospray Liquid Chromatography Native and Semi-Denaturing Mass Spectrometry

For semi-denaturing conditions, purified SOD1 was buffer exchanged into 20% acetonitrile with 0.1% formic acid (pH 2.8) using 3 kD molecular weight cutoff filters. For native conditions, SOD1 was buffer exchanged into either 50 mM ammonium acetate with 0.1% acetic acid (pH 4.5) or 100 mM ammonium bicarbonate (pH 8.0). Samples were analyzed using an Agilent 6520 Q-TOF mass spectrometer coupled to an Agilent 1260 UPLC equipped with a AdvancedBio SEC column (300 Å, 2.7 μm, 4.6×50 mm). Data was collected in positive ion mode over a range of 500-12,000 m/z. Gas temperature was 365° C. flowing at 11 L/min with a capillary voltage of 2500 V. For the highest resolution and mass accuracy, reference mass auto correction was enabled (Ref m/z 922.0098, Agilent). All data was analyzed using Masshunter B.07 with BioConfirm software for protein spectra deconvolution.

### Western Blotting

Western blot analysis was performed as described in https://doi.org/10.1016/j.ajpath.2014.08.026. Briefly, purified SOD1 WT samples were run without reducing agent and without heat treatment in Native PAGE buffer (Bio-Rad #1610738), on 12% Bis-Tris Criterion XT gels (Bio-Rad #3450118) in MOPS buffer (Bio-Rad #1610788), transferred to PVDF using a Bio Rad Trans-Blot Turbo Semi-Dry transfer apparatus (#1704150), and then probed with indicated antibodies. Mouse-anti-SOD1 antibody (BioLegend #850702, clone [O98B10]) was used at 1:2,000 dilution (0.25 μg/ml final). Subsequent incubation with horseradish peroxidase-conjugated donkey-anti-mouse IgG antibodies (Jackson ImmunoResearch #715-035-150) at 1:40,000 dilution (12.5 ng/ml final) was followed by detection with SuperSignal West Pico Plus ECL reagent (ThermoFisher Scientific #34580). ECL signals were detected with an Azure 300 Imager (Azure Biosystems). The acquired images were optimized for image quality with Adobe Photoshop version 24.3 (Adobe).

### Preparation of Human Cervical and Thoracic Spinal Cord Homogenates

Postmortem human cervical and thoracic spinal cords, with confirmed neuropathological diagnosis of sALS, SOD1 fALS, and C9ORF72 fALS and human tissue matched negative controls were received from Neil Cashman at University of British Columbia, Cindy Ly at Washington University, and the Georgetown Brain Bank. Human spinal cords were weighed to a 10% w/v homogenate using 10 mM HEPES pH 7.4 buffer. Solid tissue with buffer were put in 2 mL screw cap tubes with 1.4 mm ceramic beads (Fisherbrand) and homogenized with a Bead Mill 4 (Fisherbrand) for 60 seconds on the #4 setting. Spinal cord homogenates were subsequently spun for 5 min at 2,000×g at 25° C. Pellets were kept and supernatants were aliquoted in separate 2 mL screw cap tubes, labeled, and frozen at −80° C., and subsequently used for RT-QuIC experiments.

### Immunocapture of Misfolded SOD1 for SOD1 RT-QuIC Assay

Immunocapture was performed with ALS and negative control spinal cord tissue homogenates to control for specificity in the SOD1 RT-QuIC assay. Anti-human SOD1 antibodies O98B10 (pan-SOD1; BioLegend #850702) or C4F6 (misfolded SOD1; MédiMabs #MM-0070-2-P), and isotype control antibodies mIgG2b (ThermoFisher #MA110427) or mIgG2a (ThermoFisher #MA110418), were cross-linked to Dynabeads M-270 Epoxy (ThermoFisher #14301) according to the manufacturer's instructions. Beads were isolated with a magnet, washed with PBS, 0.025% (v/v) Tween-20 to remove unbound antibodies and blocked with PBS, 0.1% BSA for 1 hr. on a tube rotator. Prior to immunocapture, antibody-coated beads were washed once with PBS. For immunocapture, 100 μl beads (3.3×10⁷) were incubated with equal volumes (900 μl) of spinal cord homogenates (0.5% w/v in PBS) for 16 hours at 4° C. with end-over-end mixing. Flowthrough (unbound fraction) was collected and used in SOD1 RT-QuIC assay.

### SOD1 RT-QuIC Assay

SOD1 was initially expressed and purified from three different E. coli strains (T7 #C3013, T7 #2566I, and BL21 (DE3) #C2527I, New England Biolabs) and settled on using BL21 E. coli to express all SOD1 substrate for RT-QuIC. For the SOD1 RT-QuIC reaction mix, SOD1 substrate properties were examined via mass spectrometry at different pH values, and it was observed that the apo form of SOD1 below pH 5.0 (FIG. 17). Using buffers below pH 5.0, buffers that worked previously using different salts and denaturants with varying concentrations were initially examined. The concentrations of EDTA, GuHCl, 3-ME, NaCl, sodium acetate, and substrate concentration were altered, in addition to varying plate reader temperatures and plate reader shake speeds, to discover an optimal condition for a SOD1 RT-QuIC assay not requiring beads. It was shown that an optimal reaction mix is 0.6 M GuHC1, 0.02 M sodium acetate, pH 4.0, with a final ThT concentration of 15 μM, with an appropriately prepared human SOD1 substrate concentration of 50 μM (−0.9 mg/mL, 5,500 M-1 cm-1) performed at 500 rpm and 37° C. SOD1 substrate was used fresh or from aliquots frozen at −80° C. and filtered in 100 kD Pall centrifugal filter at 5,000×g, 15° C., for 15 minutes prior to mixing in RT-QuIC buffer. Solutions were made fresh with a 100 mL volumetric flask using ≥99% GuHC1 (Sigma Aldrich), 99% sodium acetate (Alfa Aesar), and high-performance liquid chromatography grade ddH2O (Alfa Aesar). The RT-QuIC reactions were performed in clear bottom 96-well microplates (Thermo-Scientific Nunc 96-well optical bottomed black polystyrene plates w/Lid, catalog #165305). Ninety-eight μL of reaction mix (15 μM ThT, 50 μM (−0.9 mg/mL) human SOD1 (filtered), 0.6 M GuHC1, 0.02 M sodium acetate, pH 4.0) were put in each well, and seeded with 10-fold dilutions of human spinal cord (cervical or thoracic) or motor cortex homogenates diluted in 1×PBS (made in-house). Plates were sealed with sealing tape (Thermo-Scientific, clear polyolefin, non-sterile, catalog #232702) and were put in FLUOstar Omega readers (BMG Labtech, Germany) at 37° C., 500 rpm, with 30 seconds of shaking and 30 seconds of resting on a double orbital setting. ThT fluorescence intensity measurements were collected every 40 minutes with 448 nm excitation and 482 nm emission using a gain setting of 1,200. Experiments were conducted using negative and positive control spinal cord and motor cortex homogenate dilutions from 10⁻² to 10⁻⁵.

### Circular Dichroism Spectroscopy

Purified SOD1 was prepared as described above, except one preparation of SOD1 was purified in the presence of β-ME (reduced, see protein purification methods), and the other SOD1 preparation contained no β-ME. Far-UV secondary structure wavelength scans were performed on 10 μM SOD1 (reduced or non-reduced during purification) in 0.6 M GuHCl, 0.02 M sodium acetate, 0.02 M ThT, pH 4.0 in a 1 cm quartz cuvette with the buffer blank subtracted. Wavelength scans were repeated multiple times, and the mean wavelength scan was used.

### Synthetic Human Recombinant SOD1 Seeds

| Purified human SOD1 WT protein was added to a solution composed of 0.6 M GuHCl, 0.02 M sodium acetate, 0.02 M ThT, pH 4.0, with final SOD1 protein concentration equal to 50 μM (~0.9 mg/mL). 150 μL of this solution was added to 96 well plates (Nunc, Thermofisher) with or without ThT and with 1.4 mm ceramic beads and put in a BMG Labtech FLUOstar Omega plate reader at 37° C. with double orbital shaking, 445 nm excitation, and 485 nm emission at 700 rpm. After maximum ThT fluorescence was observed, the plate reader was stopped, and wells containing no ThT were scraped and put into 2 mL screw capped tubes and used as synthetic human SOD1 WT seeds. The concentration of synthetic seeds was examined using a Pierce BCA Protein Assay (ThermoScientific) following manufacturer's protocol. SOD1 synthetic seeds were diluted 10-fold and in 1×PBS. Filtered human SOD1 WT substrate at 50 μM final concentration was put in RT-QuIC reaction mix (0.6 M GuHCl, 0.02 sodium acetate, 0.02 M ThT, pH 4.0) and 98 μL was added to each well in a 96 well plate and seeded with 2 μL of human SOD1 WT synthetic seeds at their appropriate dilutions.

### SOD1 RT-QuIC Assay Development and Optimization

Human SOD1 was expressed and purified from three different E. coli strains (T7 #C3013, T7 #, and BL21 (DE3) #C2527I, New England Biolabs) and was used BL21 E. coli to express all SOD1 wild type (WT) substrate for RT-QuIC. For the SOD1 RT-QuIC reaction mix, the examined properties of the human SOD1 substrate via mass spectrometry at different pH values and was observed the apo form of SOD1 below pH 5.0 (FIG. 17). The concentrations of EDTA, GuHCl, βME, NaCl, sodium acetate, and the substrate were altered, in addition to varying plate reader temperatures and plate reader shake speeds, to discover an optimal condition for a SOD1 RT-QuIC assay not requiring beads. It was discovered that an optimal reaction mix is 0.6 M GuHCl, 0.02 M sodium acetate, pH 4.0, with a final ThT concentration of 15 μM, with an appropriately prepared human SOD1 substrate concentration of 50 μM (~0.9 mg/mL, 5,500 M⁻¹ cm⁻¹) performed at 500 rpm and 37° C. human SOD1 substrate was used fresh or from aliquots frozen at −80° C., and filtered in 100 kD Pall centrifugal filter at 5,000×g, 15° C., for 15 minutes prior to mixing in RT-QuIC buffer. Solutions were made fresh with a 100 mL volumetric flask using ≥99% GuHCl (Sigma Aldrich), 99% sodium acetate (Alfa Aesar), and high-performance liquid chromatography grade ddH₂O (Alfa Aesar). The RT-QuIC reactions were performed in clear bottom 96-well microplates (Thermo-Scientific Nunc 96-well optical bottomed black polystyrene plates w/Lid, catalog #165305). Ninety-eight μL of reaction mix (15 μM ThT, 50 μM (~0.9 mg/mL) human SOD1 (filtered), 0.6 M GuHCl, 0.02 M sodium acetate, pH 4.0) were put in each well, and seeded with 10-fold dilutions of cervical or thoracic human spinal cord homogenates diluted in 1×PBS (made in-house). Plates were sealed with sealing tape (Thermo-Scientific, clear polyolefin, non-sterile, catalog #232702) and were put in FLUOstar Omega readers (BMG Labtech, Germany) at 37° C., 500 rpm, with 30 seconds of shaking and 30 seconds of resting on a double orbital setting. ThT fluorescence intensity measurements were collected every 40 minutes with 448 nm excitation and 485 nm emission using a gain setting of 1,200. Experiments were conducted using negative and positive control human spinal cord homogenate dilutions from 10⁻² to 10⁻⁵.

### Transmission Electron Microscopy of Converted or Non-Converted RT-QuIC Products

Converted and non-converted SOD1 RT-QuIC products were negatively stained using 3% aqueous phosphotungs (Stic acid (PTA). Briefly, samples were vortexed and adhered to glow-discharged ultrathin carbon on lacey carbon 400 mesh copper grids (Electron Microscopy Sciences) for 1 minute. Samples were lightly blotted, followed by ddH₂O rinse, lightly blotted, and finally stained with 3% PTA. Grids were imaged in a HT7800 (Hitachi) transmission electron microscope operating at 80 kV. Micrographs were acquired on an XR-81 camera (Advanced Microscopy Techniques).

### RT-QuIC Statistical Analysis

ThT relative fluorescence units (RFU) versus time were collected with Omega Mars software synced to the microplate readers, then raw data was exported to Microsoft Excel and graphed and analyzed in SigmaPlot 14.5 or 15.0 (Systat software). All RT-QuIC reactions were done in at least quadruplicate for ALS patient tissues and control tissues at 10⁻³, 10⁻⁴, and 10⁻⁵ tissue dilutions. ThT relative fluorescence units (RFU) versus time was plotted for each ALS patient tissue homogenate and each human negative control tissue homogenate at their equal tenfold tissue dilutions. It was observed that the kinetic curves approached stationary phase, but did not entirely plateau, for some ALS spinal cord dilutions. Thus, it was then fitted raw curves to equation 1 to extract RT-QuIC parameters.

In equation 1,

$$y = {y}_{0} + \frac{A}{( 1 + {e}^{- ( \frac{x - {x}_{0}}{τ} )} )} \qquad ( 1 )$$

where y₀ is initial ThT fluorescence, A is ThT amplitude, x₀ is time to reach 50% ThT fluorescence, and τ is a fibril time constant. The time to fluorescence positivity threshold or lag phase is equal to x₀−2τ. Raw data was fitted to this model for each kinetic curve, to estimate x₀, y₀, τ, and A, which is the difference between final and initial ThT fluorescence. When time (x) equals x₀, equation 1 reduces to equation 2.

$${y}_{50} = {y}_{0} + \frac{A}{2} \qquad ( 2 )$$

Used was y₀ and A to determine y₅₀ values (i.e., 50% ThT fluorescence), and used τ and x₀ to calculate lag phase (x₀−2τ). Following the experiment, plotted mean 50% ThT fluorescence (RFU) versus lag phase (hours) to examine correlation between these two variables. After graphing 50% ThT fluorescence versus lag phase for each ALS patient cord at 10⁻³, 10⁻⁴, and 10⁻⁵ dilution, used was equation 3 to examine dependence of 50% ThT fluorescence on lag phase and determine the linear correlation coefficient R. In equation 3,

$$y = β + m \times α \qquad ( 3 )$$

where β is y-intercept, m is slope, and a is lag phase.

### Cutoff for Negative and Positive Samples Using Lag Phase and ThT Fluorescence Amplitude

All RT-QuIC reactions were performed in quadruplicate for negative controls and ALS patient samples at several 10-fold tissue dilutions. Thioflavin T fluorescence is observed in human negative control spinal cords at 10⁻², but this ThT fluorescence is no longer observed past 10⁻², indicating some non-specific ThT fluorescence at more concentrated dilutions of human spinal cord. The ALS samples, however, remain ThT positive past 10⁻², and continue to remain ThT positive to 10⁻⁵, indicating these samples have misfolded form(s) of human SOD1. Human negative control spinal cords either had no ThT amplitude or had kinetic separation of ~120 hours between ALS samples at 10⁻³ spinal cord dilutions.

SOD1 RT-QuIC Assay with Human Cerebrospinal Fluid (CSF)

CSF was prepared in 1 part CSF to 5 total parts (or 1 part CSF to 10 total parts) of high performance liquid chromatography water (Sigma Aldrich), and 2 microliters was added to each well following SOD1 RT-QuIC assay as previously described above.

### RESULTS

### Properties of Human Non-Mutant SOD1 Substrate for RT-QuIC

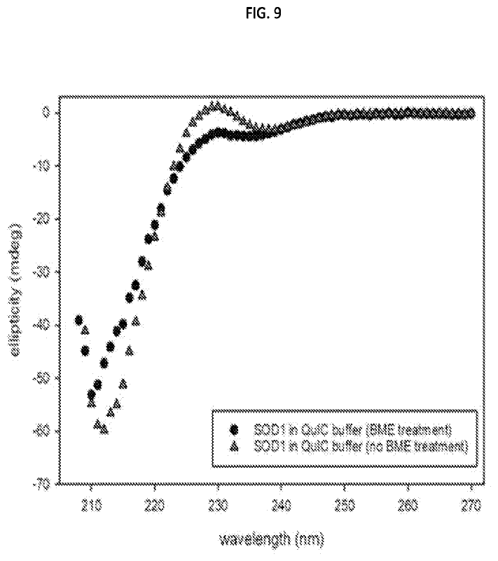

RT-QuIC assays require non-fibrillar substrate protein molecules that can be recruited into growing fibrils in the presence of pre-existing ex vivo seeds more rapidly than they spontaneously nucleate into seeding-competent assemblies under assay conditions. Such non-fibrillar substrates are not necessarily identical to the physiological forms of the given protein and its thermodynamic stability. Because native non-mutant SOD1 contains two free cysteine thiols and one intramolecular disulfide bond per monomeric unit, histidine-tagged wild-type human SOD1 (recombinant SOD1; rSOD1) was grown in BL21 E. coli and purified in the presence or absence of the reducing agent β-ME using metal-ion affinity chromatography. Non-reducing SDS-PAGE gels of eluted fractions exposed to SDS without heating indicated the presence of rSOD1 monomers and dimers in a major peak (pk2) of eluted protein (FIG. 6). After dialysis, native PAGE gels and immunoblotting with anti-SOD1 antibodies indicated that in both a Tris buffer and RT-QuIC reaction buffer, rSOD1 was primarily monomeric (FIG. 7). The mass of rSOD1 prepared with or without β-ME was confirmed, with the presence of β-ME allowing formation of an intramolecular disulfide bond (FIG. 17). This disulfide was not present in preparation of rSOD1 without β-ME (FIG. 17), suggesting E. coli did not form the disulfide bond in rSOD1. Using electrospray ionization LC-MS, it was examined that the metal occupancy of rSOD1 at different pHs. This analysis indicated the apoenzyme predominated at low pH (pH 2.8) while copper was likely bound at higher pH (pH 5.5) (FIG. 17). Circular dichroism spectroscopy in the far-UV region showed rSOD1 prepared with or without β-ME differed in secondary structure (FIG. 9). Reduced ellipticity at 230 nm suggested a loss of polyproline II helix secondary structure in the β-ME-treated rSOD1 in RT-QuIC buffer where the CD spectrum is random coil; which was not observed with rSOD1 in RT-QuIC buffer prepared without β-ME treatment. In summary, these data provided evidence that β-ME-treated rSOD1 substrate used in RT-QuIC assays below was largely monomeric (FIG. 7), contains one intramolecular disulfide bond (FIG. 17), is random coil (FIG. 9), and is metal-free (FIG. 17).

Seeding of rSOD1 Fibrillization by ALS Spinal Cord Homogenates in RT-QuIC Conditions

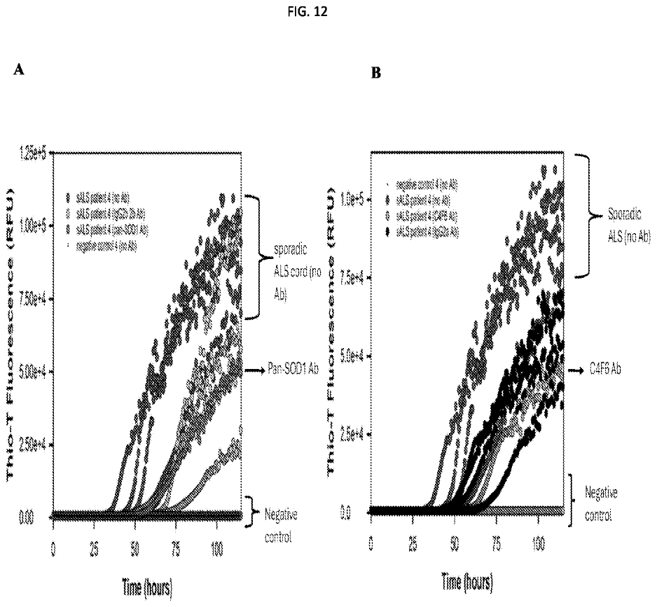

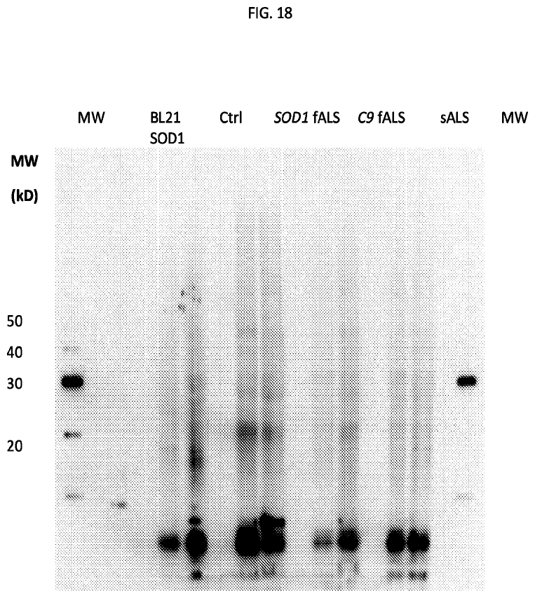

Given SOD1 pathology is commonly observed in spinal cord of SOD1 fALS patients, initial confirmation of the presence of abnormal SOD1 in spinal cord homogenates from six ALS cases and two human negative controls by western blotting using a pan-SOD1 antibody were sought. Although the control spinal cords gave a broad smear of bands above the size of the SOD1 monomer, spinal cords from patients with SOD1 fALS, sALS, or C9ORF72 fALS, tended to have enhanced intensities and distinct patterns of larger bands suggestive of greater abundance of higher-order structures (FIG. 18). SOD1 RT-QuIC assay was then used to discern clear kinetic discrimination of SOD1 seeding activity between ALS patient cord homogenates and negative control cord homogenates. For example, seeding with a 5×10⁻³ dilution of a sALS spinal cord homogenate gave enhanced ThT fluorescence in ~32-55 h, while controls remained negative for >100 h (FIG. 12). These results provided initial evidence of sALS-associated SOD1 RT-QuIC seeding activity.

Immunodepletion of sALS-Associated Seeding Activity with Anti-SOD1 Antibodies

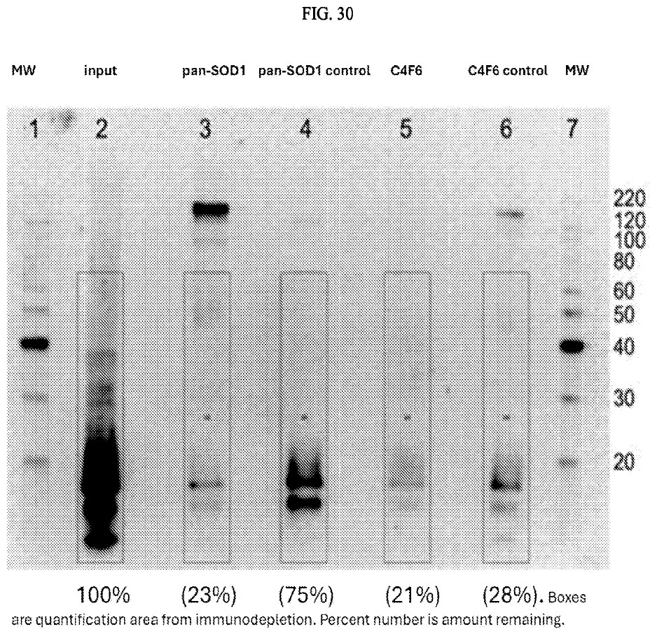

The SOD1 specificity of the assay was investigated by performing immunodepletion experiments. A sALS cord homogenate was either left untreated or incubated with magnetic beads cross-linked to the following antibodies: an antibody to misfolded SOD1 (C4F6), a pan-SOD1 antibody, or two isotype-matched control antibodies. The beads were removed, and the remaining spinal cord supernatants assayed by SOD1 RT-QuIC. It was observed that a greater reduction in SOD1 seeding activity with SOD1 antibodies (C4F6 and a pan-SOD1 antibody) than with isotype-matched control antibodies (FIG. 12), providing evidence that a least some of the seeding activity in the sALS spinal cord contained SOD1 (Table 1). Immunoprecipitation experiments with the sALS spinal cord homogenate showed a greater reduction of SOD1 protein when captured with SOD1 specific antibodies relative to capture using isotype control antibodies (FIG. 30).

TABLE 1
SOD1 RT-QUIC parameters for immunedepletion
of sALS spinal cord homogenate

| | Lag phase | 50% ThT |
|---|---|---|
| Sample | (hours) | (RFU) |
| sALS patient 4 (no Ab) | 48.37 ± 11.03 | 43,500 ± 5467 |
| sALS patient 4 (pan-SOD1 Ab) | 62.68* | 27,293* |
| sALS patient 4 (pan-SOD1 isotype | 70.22 ± 6.803\hat{ } | 32,100 ± 19,029\hat{ } |
| control Ab) | | |
| sALS patient 4 (C4F6 Ab) | 62.41* | 21,878* |
| sALS patient 4 (C4F6 isotype | 57.56 ± 10.35\hat{ } | 27,600 ± 6,406 |
| control antibody) | | |

*= ¼ wells ThT positive,
\hat{ }= ¾ wells ThT negative

### Human SOD1 Synthetic Fibrils Propagate Human SOD1 Substrate

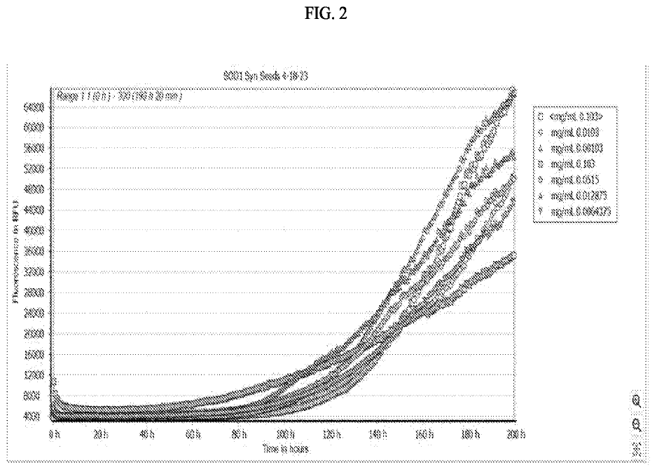

The concentration of purified human SOD1 WT substrate in 25 mM Tris pH 8.0 was measured, and then diluted to 150 μM (~2.0 mg/mL) in the RT-QuIC buffer with or without 20 μM Thioflavin T (ThT). Approximately 200 μL of this reaction mix was put in a 96 well plate and shaken at 37° C. at 700 rpm with one 1.4 mm ceramic bead in each well. Once ThT fluorescence reached stationary phase, the plate reader was stopped and each well without ThT was scraped and pooled into a screw cap tube then concentrated in a 10,000 MWCO Amicon. A BCA assay was run on human SOD1 WT synthetic protein seeds to determine stock protein concentration, and these synthetic human SOD1 WT seeds were diluted 10-fold in 1×PBS and used in an RT-QuIC experiment using fresh human SOD1 WT substrate as described (see methods). In vitro SOD1 synthetic seed experiment suggests that this human SOD1 WT substrate can successfully propagate misfolded SOD1 WT (FIG. 2).

SOD1 Seeding Activity in SOD1 fALS, C90RF72 fALS, Sporadic ALS Patient Spinal Cords

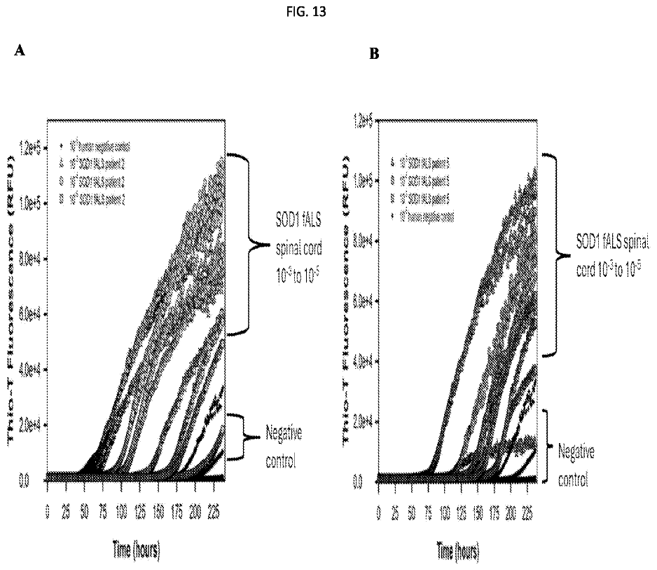

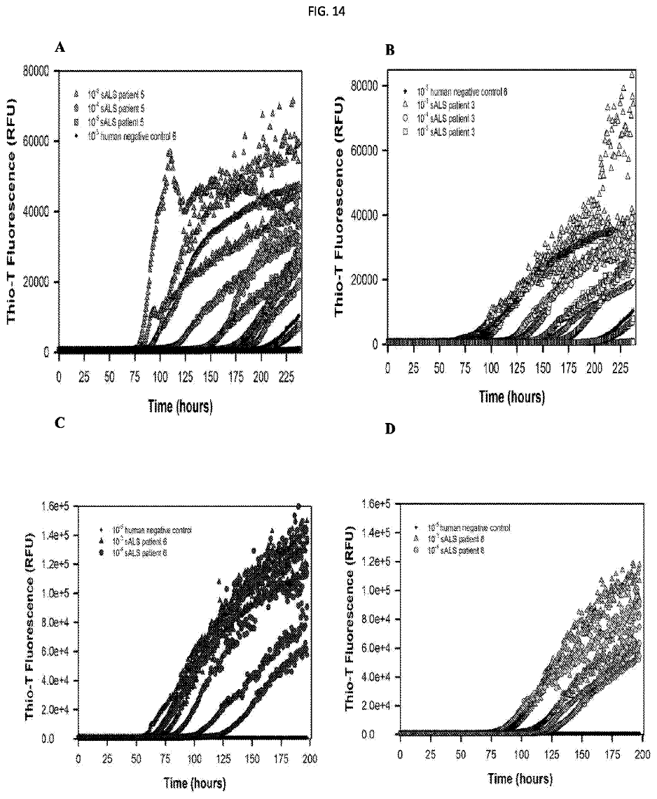

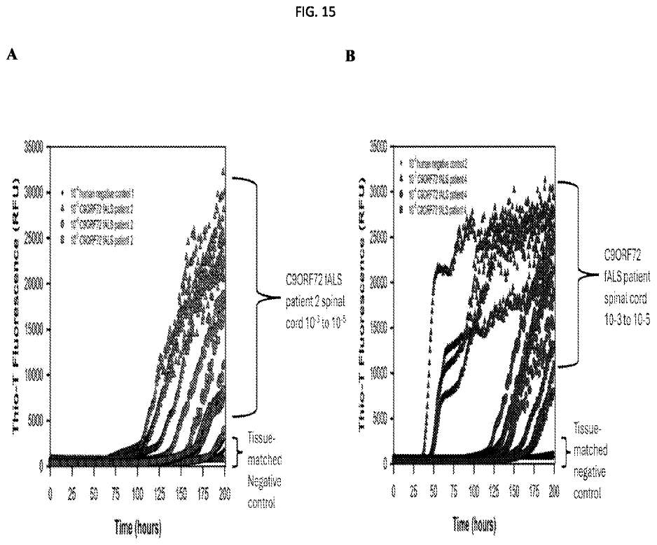

The study was expanded by comparing spinal cord tissue from patients clinically diagnosed with sALS (n=10), fALS linked to mutations in SOD1 (n=5) or C90RF72 (n=5), or human negative controls (n=12). For 15 of the 20 total ALS patients, cervical spinal cord was used. For sALS cases, five cervical and five thoracic spinal cord specimens were analyzed. It was then homogenized spinal cords at 10% w/v and assayed 10-fold serial dilutions thereof. At 10⁻², it was then observed matrix inhibition of ThT fluorescence in several ALS spinal cord homogenates, but at 10⁻³, 10⁻⁴, and 10⁻⁵ tissue dilutions, the sALS, SOD1 fALS, and C9ORF72 fALS spinal cords usually gave enhanced ThT fluorescence prior to that elicited by the negative control spinal cords (FIGS. 13-16). The sALS C9ORF72-linked fALS cervical cord specimens gave substantially lower ThT fluorescence intensity relative to those elicited by SOD1 fALS and sALS thoracic cords at same dilutions. Collectively, these data provide evidence that SOD1 RT-QuIC can detect SOD1 seeds in neural tissue of patients with both sporadic and genetic etiologies of ALS.

### SOD1 Seeding Activity in Thoracic Cord and Motor Cortex of Sporadic ALS Patients

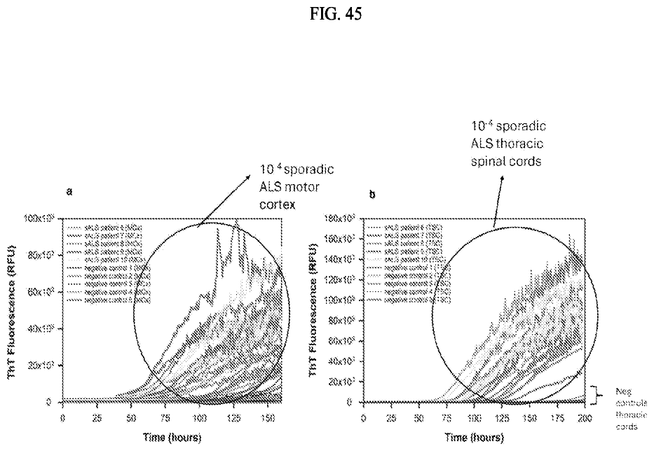

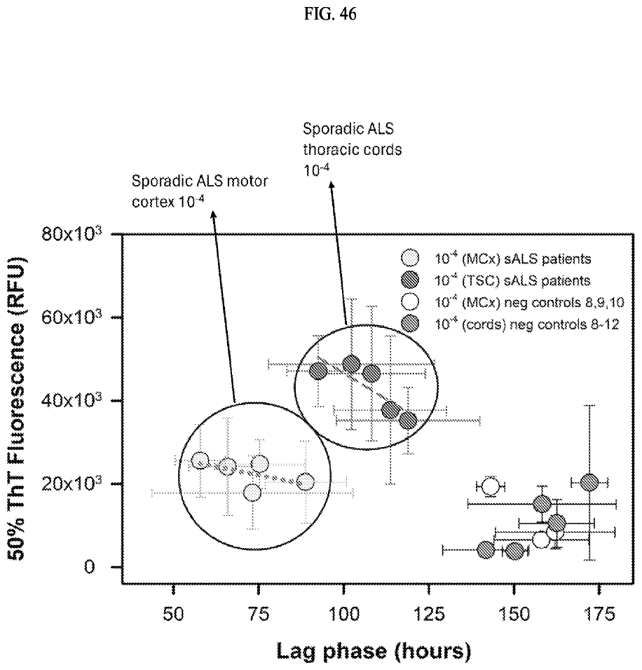

Because ALS pathology can have neurodegeneration of upper and lower motor neurons, the medial primary motor cortex of five sporadic ALS patients was further examined and compared these seeding activity results to their thoracic cord (Table 2, sALS patients 6, 7, 8, 9, and 10) and to eight tissue-matched non-ALS negative controls (neurological and non-neurological, Table 2). Medial primary motor cortex was homogenized at 10% w/v and run in SOD1 RT-QuIC by seeding wells with brain dilutions from 10⁻² to 10⁻⁵. At 10⁻² and 10⁻³, it was observed that no ThT fluorescence, probably because of motor cortex matrix inhibition of ThT, however at 10⁻⁴ and 10⁻⁵ tissue dilutions, we observed substantial ThT fluorescence in all five sporadic ALS patient's motor cortex relative to non-ALS motor cortex controls (FIG. 45). The SOD1 seeding activity comparison between these two anatomical regions in each sporadic ALS patient suggests their motor cortex lag phase is shorter than that from their thoracic cord (FIG. 46).

Detecting Misfolded Forms of Human Superoxide Dismutase 1 in Postmortem Human Spinal Cords with Familial SOD1 or Sporadic Amyotrophic Lateral Sclerosis Using a Superoxide Dismutase RT-QuIC Assay

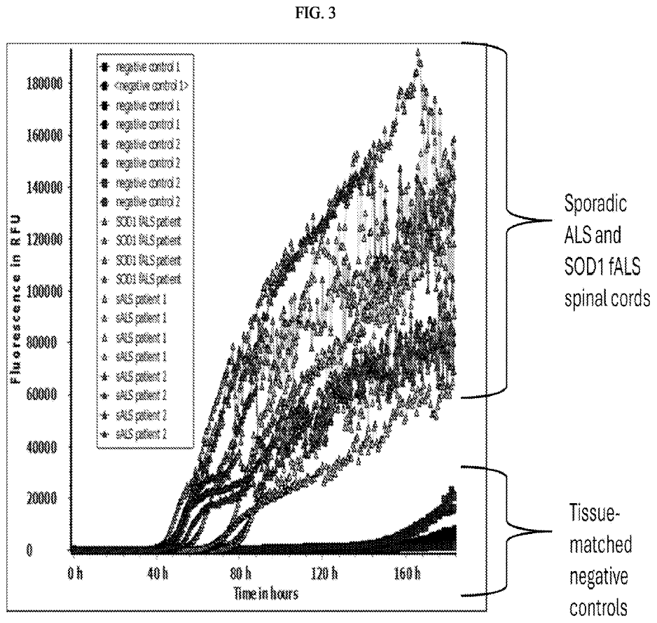

After establishing conditions that are suitable for the detection of misfolded SOD1, the 10% w/v homogenized human spinal cords was diluted at several 10-fold dilutions in 1×PBS containing 98 μL of the SOD1 RT-QuIC reaction mix at each tissue dilution. Plots of ThT RFU versus time are shown for SOD1 fALS and sALS human spinal cords (FIG. 3) and versus two human negative control spinal cords at 10⁻³ dilutions. Kinetic separation of approximately 120 hours is observed between ThT positive and ThT negative samples. Thus, the results in FIG. 3 suggest misfolded form(s) of SOD1 protein are in SOD1 fALS and sALS patient spinal cords and can be detected using the SOD1 RT-QuIC assay at high sensitivity and specificity.

### Electron Microscopy of SOD1 RT-QuIC Products

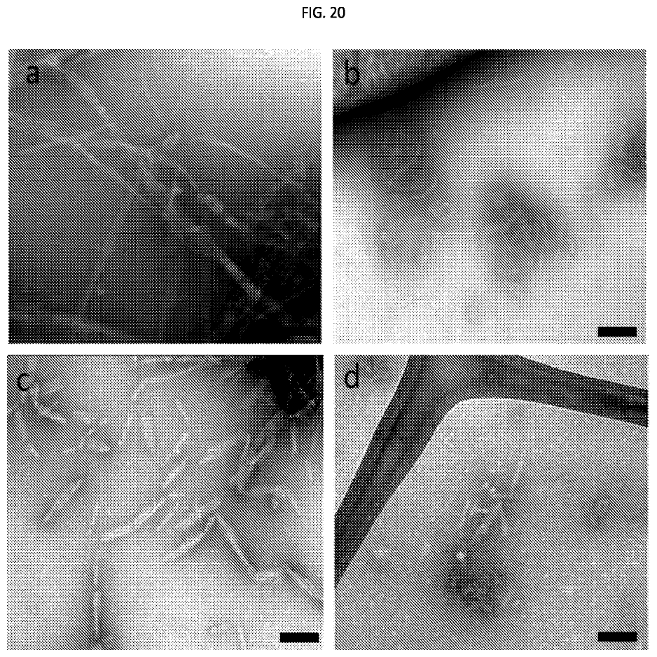

ThT positivity of amplified ALS-seeded SOD1 RT-QuIC kinetic curves suggested they were amyloid fibrils. To confirm this visually, ThT-positive products of SOD1 RT-QuIC reactions seeded with sALS and fALS spinal cord homogenates by negative stain transmission electron microscopy were analyzed and it was found that they had abundant fibrils (FIG. 20). Fibrils were also observed in reactions initiated with negative control cord homogenates when the latter were allowed to incubate long enough to become ThT-positive, presumably because of eventual spontaneous nucleation of rSOD1 fibrillization.

Detecting Misfolded Forms of Human Superoxide Dismutase 1 in Postmortem Spinal Cords with Familial C90RF72 Amyotrophic Lateral Sclerosis

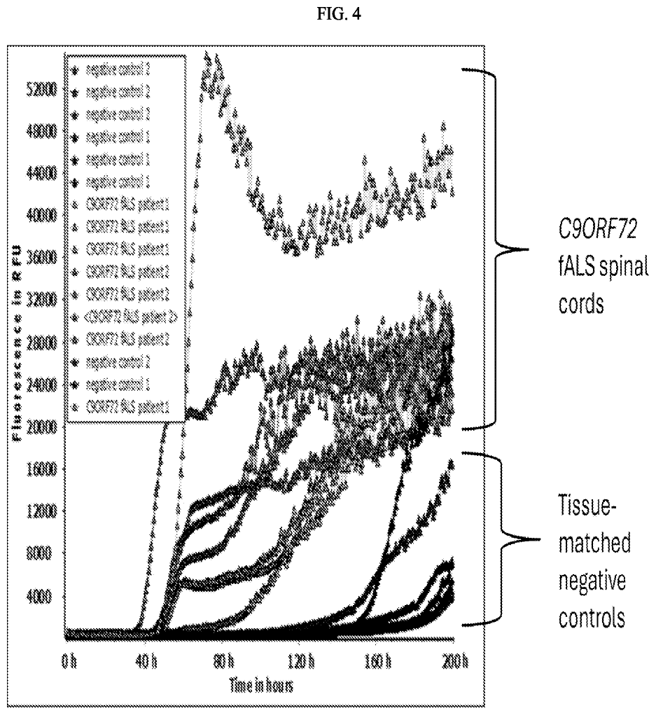

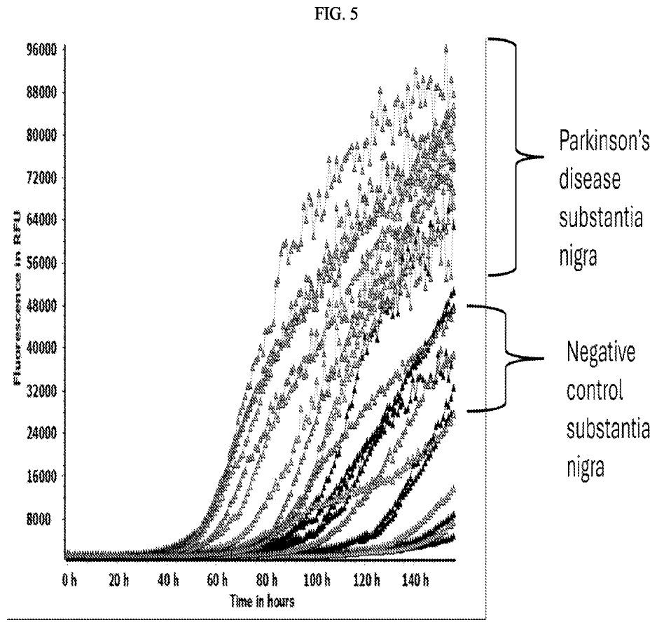

Further examined was C9ORF72 familial ALS patients for misfolded SOD1 using the described SOD1 RT-QuIC assay. Detected and propagated was the misfolded forms of SOD1 in human fALS patient spinal cords with an abnormal hexanucleotide repeat expansion in their C90RF72 gene, suggesting this type of ALS may also have SOD1 retinopathy. FIG. 4 shows two C90RF72 fALS patient spinal cords plotted with two human negative control spinal cords run in the same plate, also with a kinetic separation of ~120 hours. Taken together, data from FIGS. 3 and 4 suggest all three types of ALS (sALS, SOD1 fALS, and C9ORF72 fALS) have SOD1 misfolding that is detected with the SOD1 RT-QuIC assay. Based on these results, it was hypothesized that there may be less SOD1 seeding occurring in C9ORF72 fALS relative to SOD1 fALS and sALS patients. Thus, FIGS. 3 and 4 demonstrate that forms of misfolded SOD1 protein is present in multiple types of human ALS spinal cords, and thus may be a general biomarker for ALS using cerebrospinal fluid or other antemortem tissues.

### Achieving High Sensitivity and Specificity for Detecting Misfolded Forms of Human Superoxide Dismutase 1 Using SOD1 RT-QuIC Assay

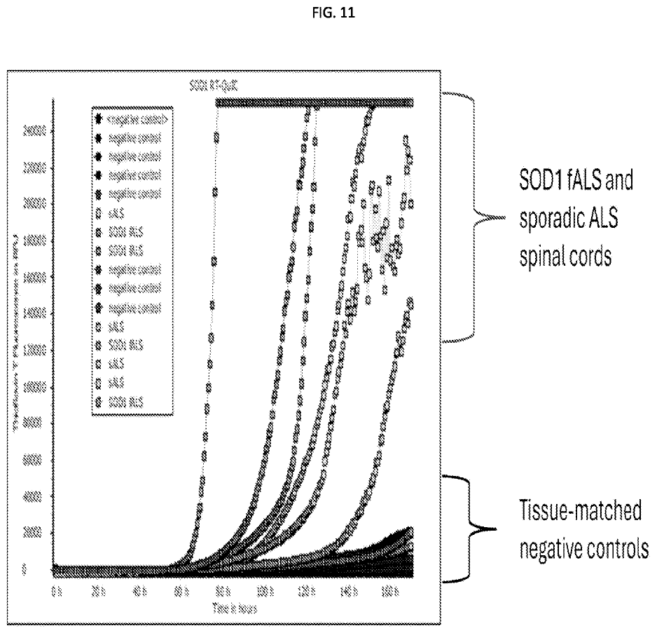

To determine whether the described SOD1 RT-QuIC assay could have relevance to important clinical applications for Amyotrophic Lateral Sclerosis and other human diseases that may have SOD1 proteinopathy, it was determined that the endpoint dilution of the SOD1 RT-QuIC assay with patient spinal cord tissues to examine sensitivity and specificity at less dilute solutions of spinal cord. FIG. 11 shows the detection of misfolded SOD1 with high sensitivity and specificity at a 10⁻⁵ spinal cord dilution from a SOD1 fALS and sALS patient.

ThT Positivity of Amplified ALS-Seeded SOD1 RT-QuIC Kinetic Curves Suggested they are Amyloid Fibrils

ThT-positive products of SOD1 RT-QuIC reactions seeded were analyzed with sALS and fALS spinal cord homogenates by negative stain transmission electron microscopy and found abundant fibrils (FIG. 20). Fibrils were also observed in reactions initiated with negative control cord homogenates when the latter were allowed to incubate long enough to become ThT-positive, presumably because of eventual spontaneous nucleation of rSOD1 fibrillization.

### Analysis of SOD1 RT-QuIC Parameters

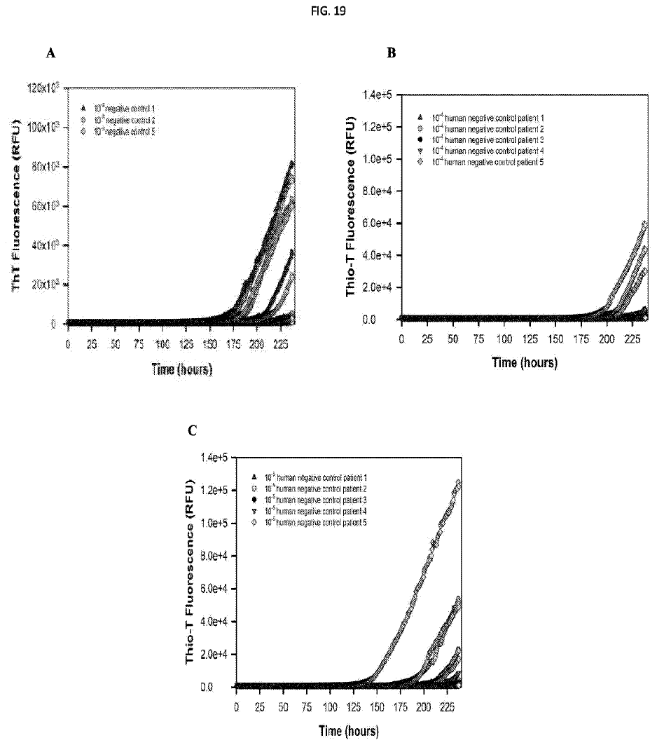

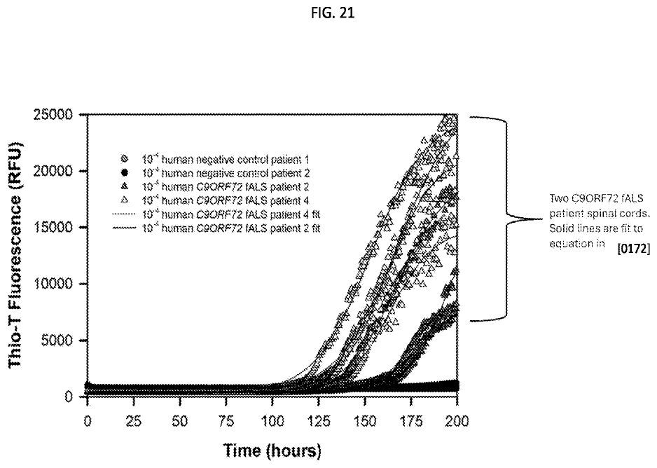

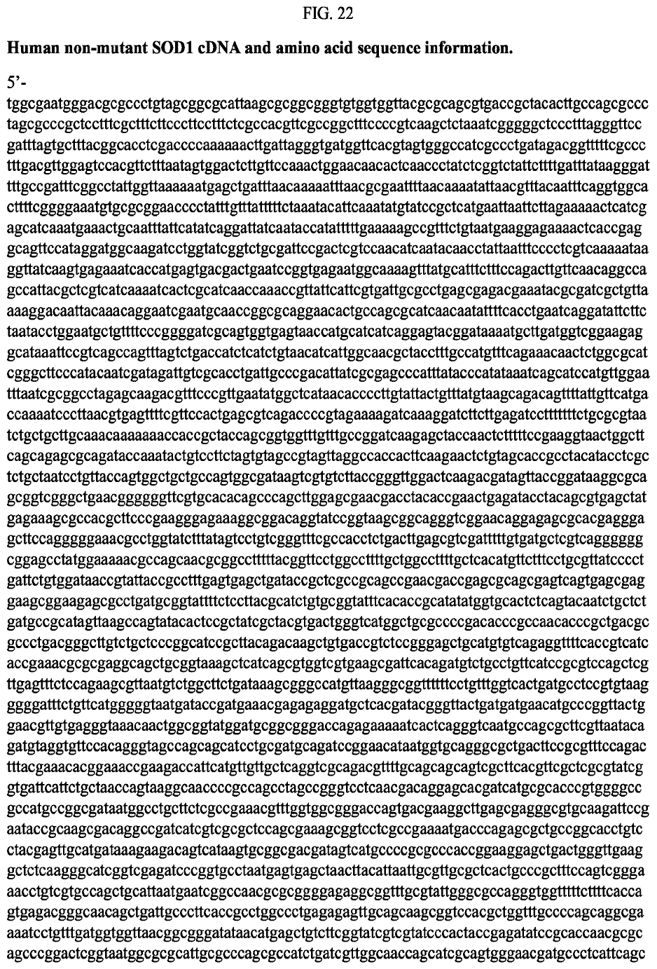

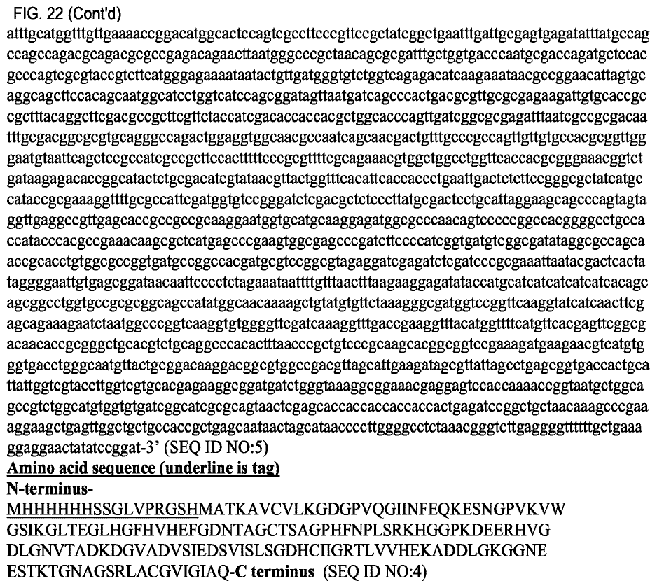

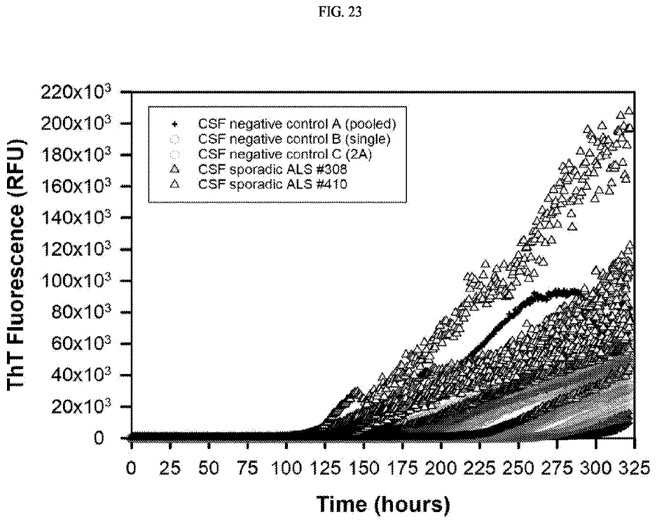

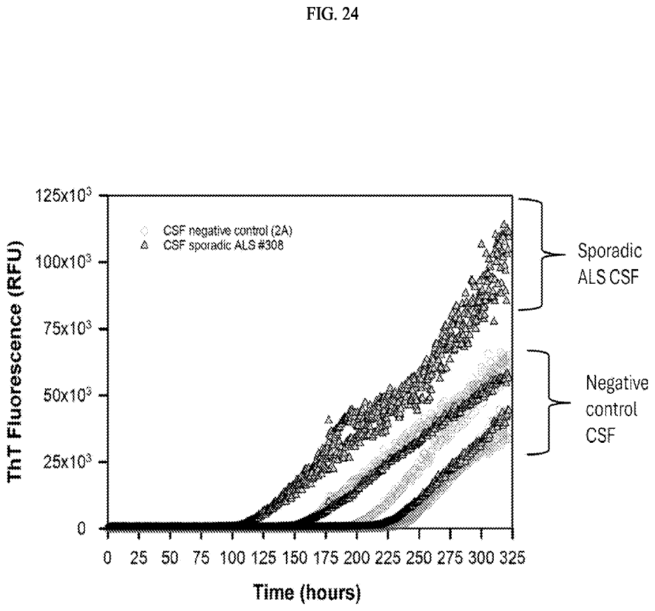

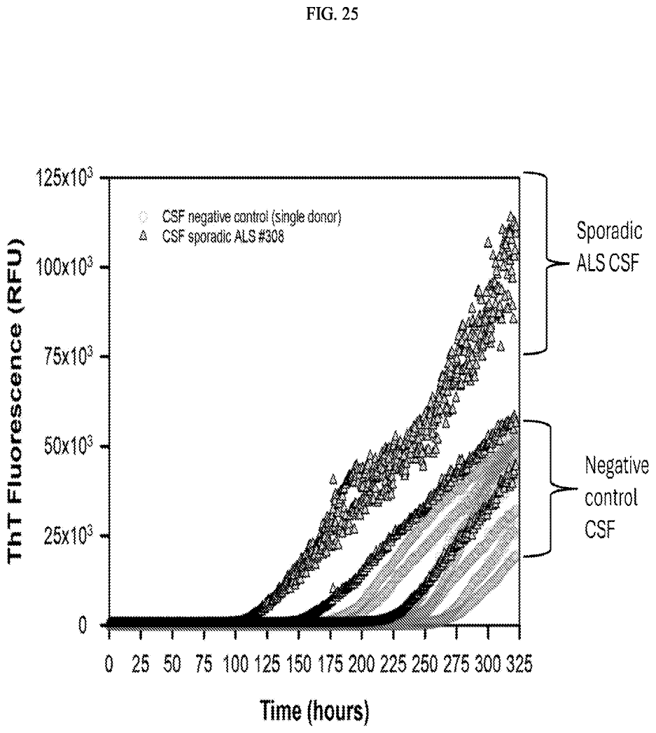

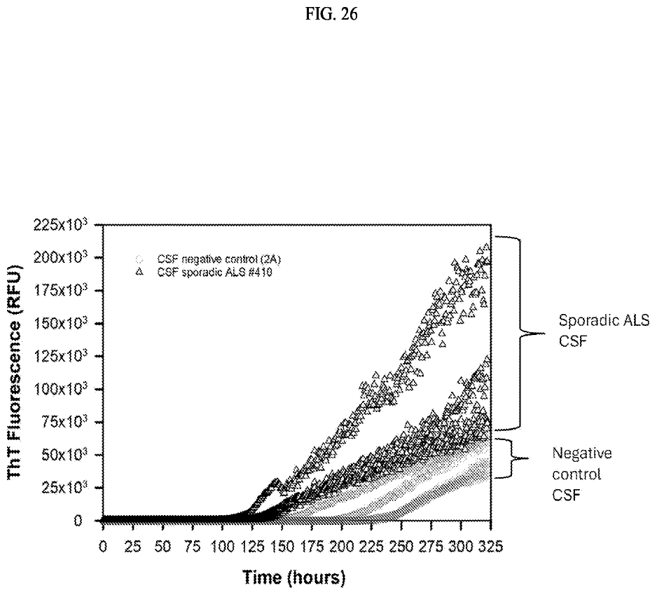

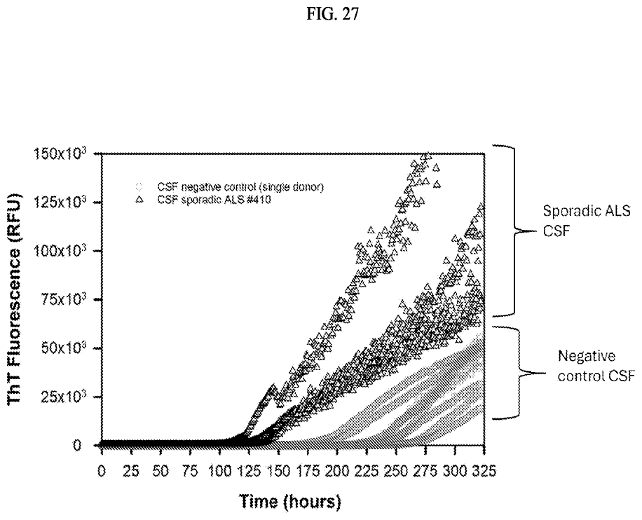

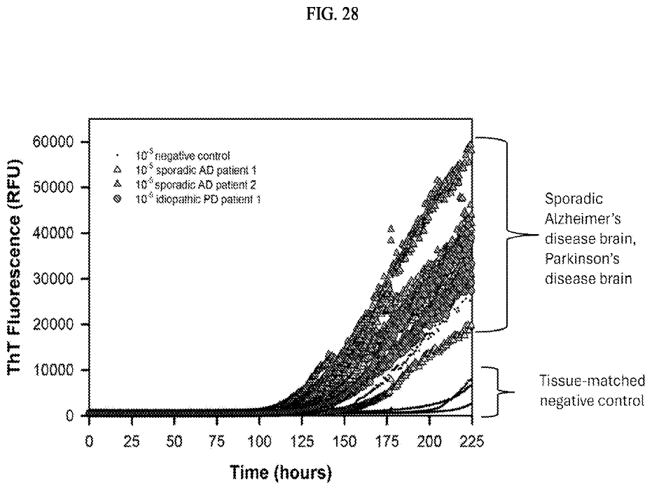

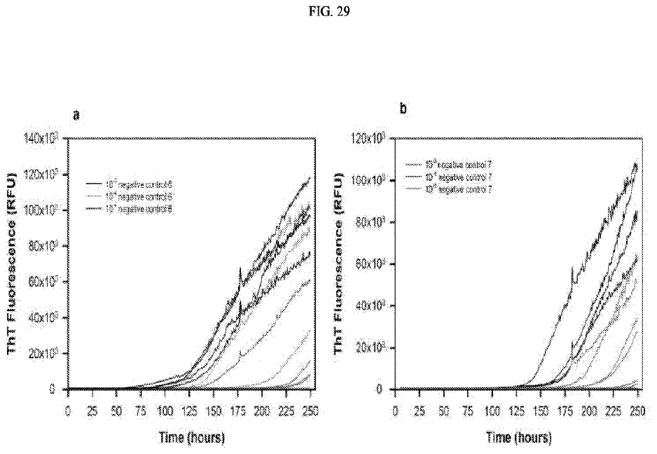

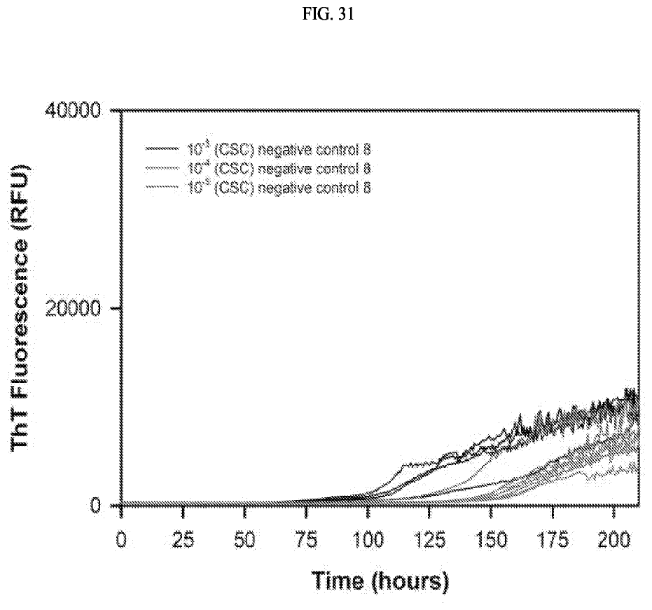

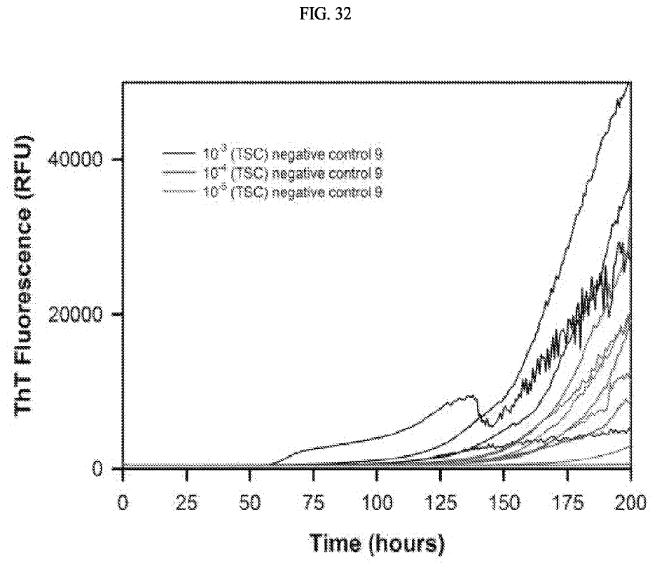

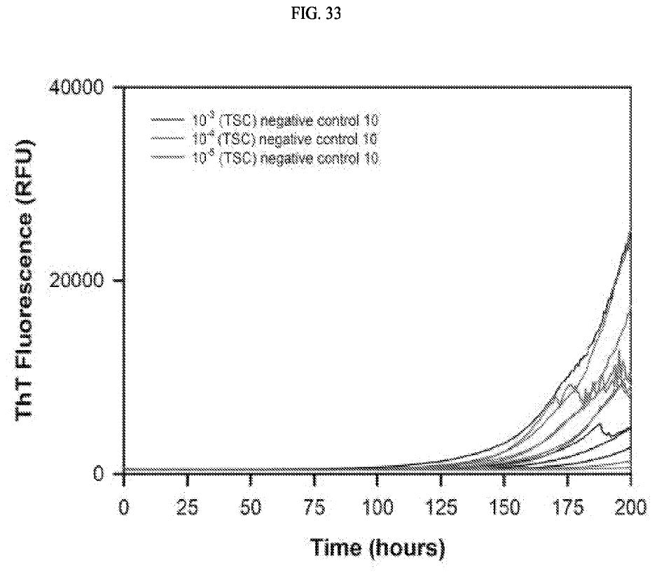

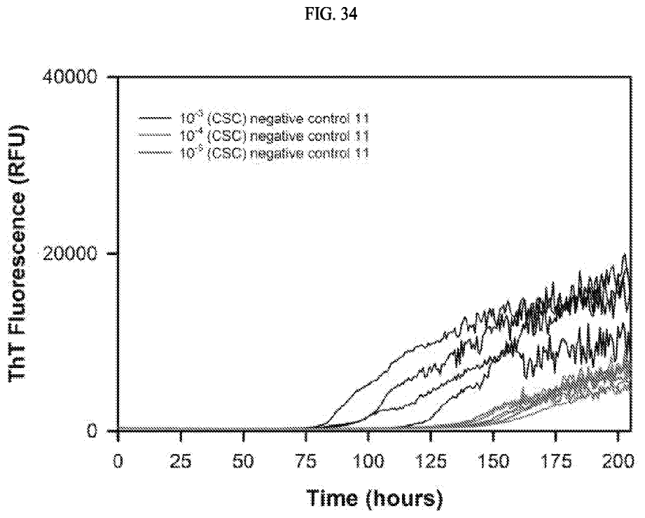

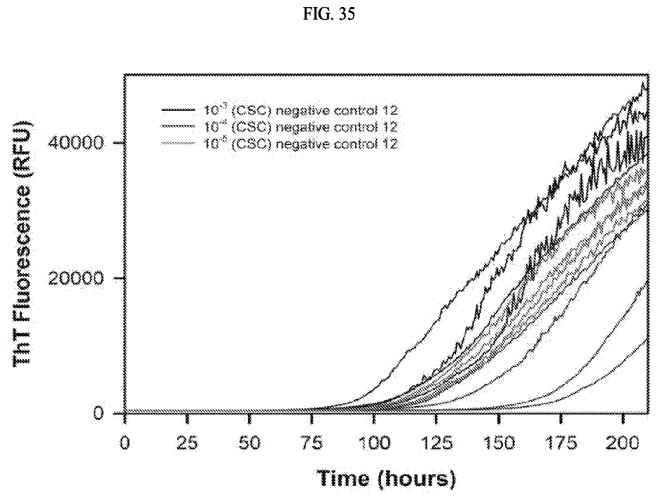

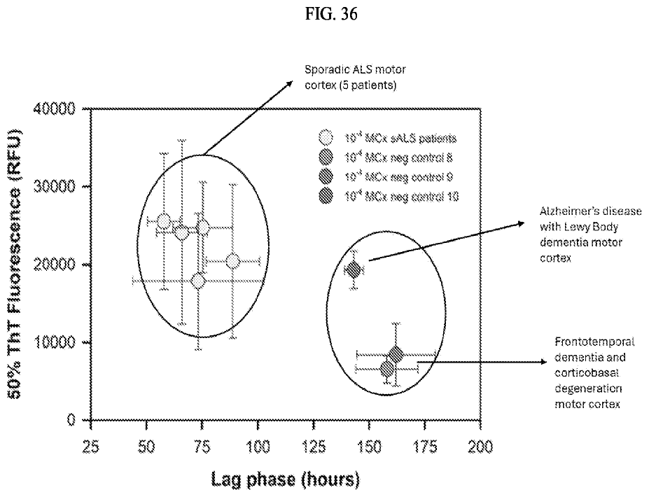

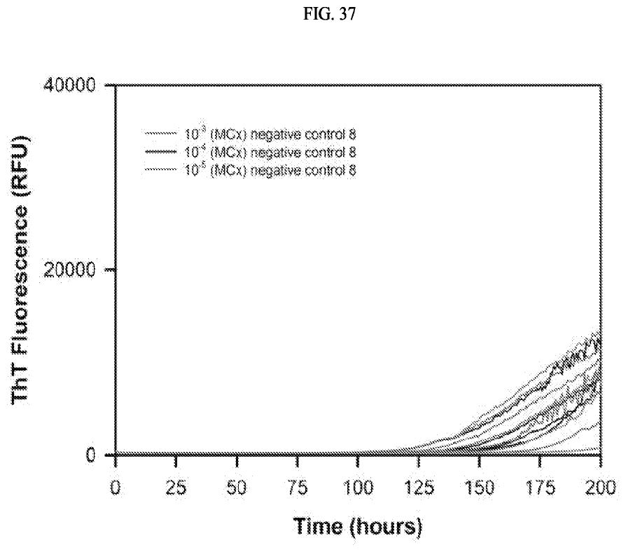

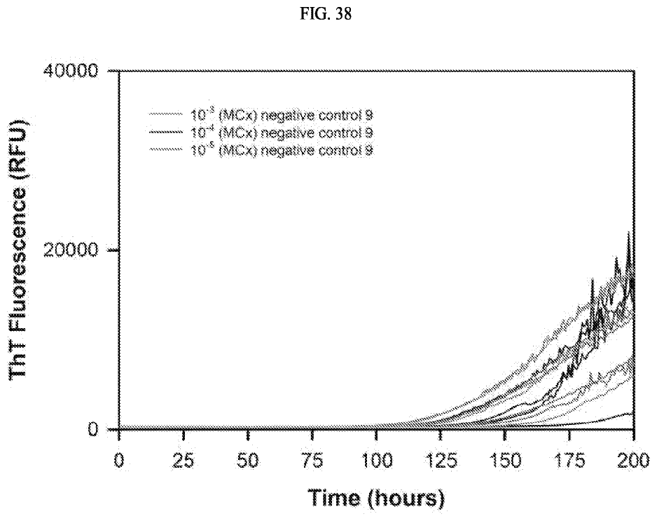

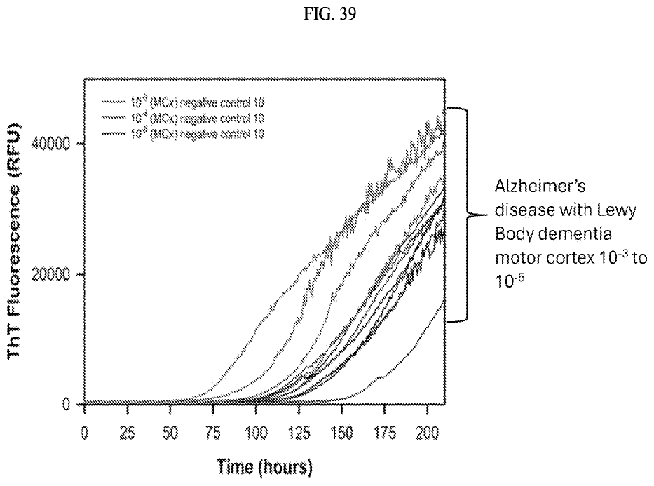

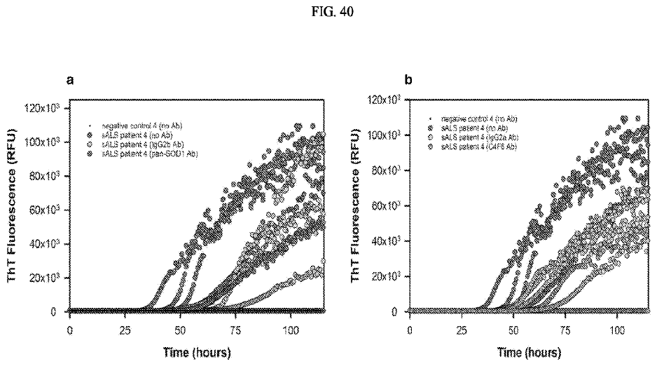

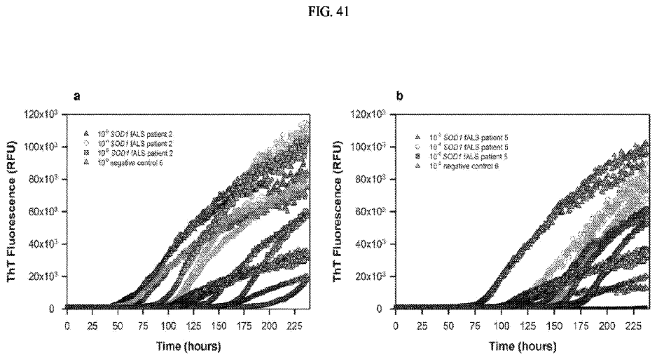

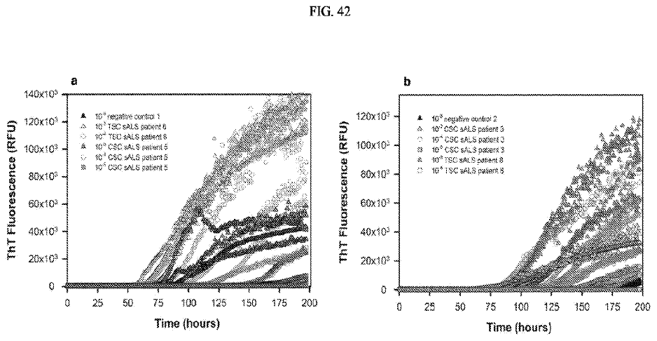

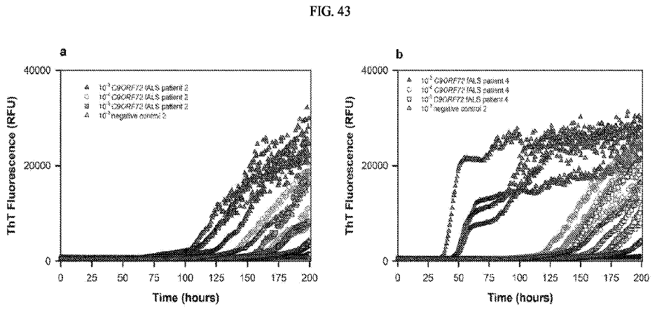

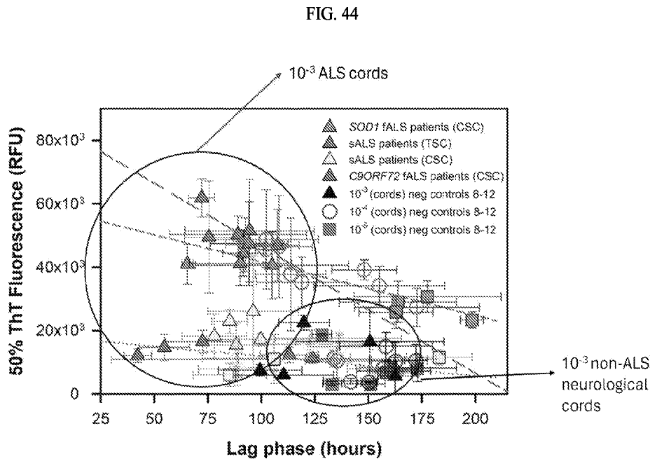

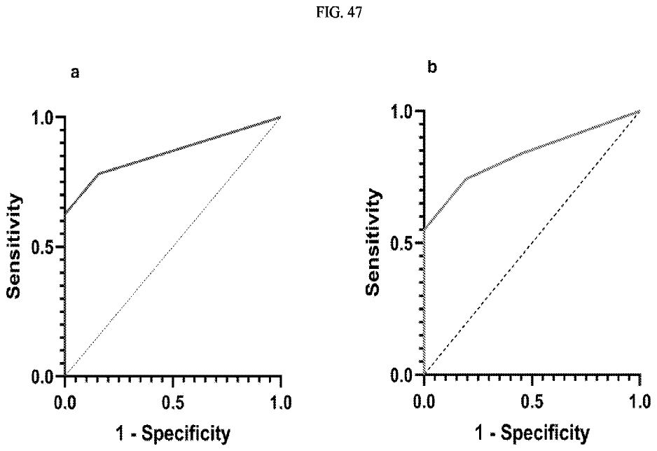

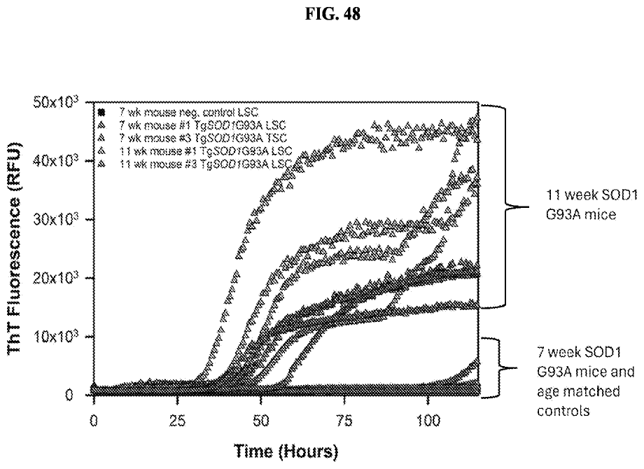

When SOD1 RT-QuIC experiments ended, it was fitted to each kinetic curve to extract its ThT fluorescence amplitude, time to 50% ThT fluorescence, initial ThT fluorescence, and a fibril time constant. FIG. 21 shows fit to the kinetic data. The parameters were used to calculate 50% ThT fluorescence and lag phase (time to a designated threshold of positive fluorescence), as previously described. For each ALS spinal cord dilution, the mean was calculated±standard deviation of lag phase and 50% ThT fluorescence and plotted these two variables for each serial spinal cord dilution. In FIG. 44, it was observed in general that 50% ThT fluorescence decreases, and lag phase extends, as ALS spinal cords are diluted from 103 to 105. It was then fit to each ALS spinal cord type (SOD1 fALS, sALS thoracic, sALS cervical, C90RF72 fALS) to equation 3, which yielded high linear correlation coefficients for SOD1 fALS cervical cords (R=0.88), C9ORF72 fALS cervical cords (R=0.85), and sporadic ALS thoracic spinal cords (R=0.84). For motor cortices from sporadic ALS patients, it a similar relationship between 50% ThT vs. lag phase was observed (FIG. 46, R=0.57). Thus, lag phase and 50% ThT fluorescence are strongly negatively correlated in most of these ALS patient neural tissues, which likely is dependent on SOD1 seed concentration (Table 3 and FIGS. 44 and 46). Most non-neurological ALS control spinal cords at these dilutions failed to elicit ThT fluorescence until −175 hours at 10⁻³ dilution and −200 hours for 10⁻⁴ and 10⁻⁵ cord dilutions (FIG. 19). Negative control 6 (FIG. 29) gave positive ThT fluorescence at −125 hours at 10⁻³ dilution, while at 10⁻⁴ and 10⁻⁵ dilutions, negative controls 6 and 7 gave ThT positivity at −160 hours and >200 hours respectively. To better understand the specificity of our SOD1 RT-QuIC assay, neurological tissue-matched specimens from patients clinically diagnosed with other neurological diseases were examined (FIGS. 31-39, 46; Table 2). The mean ThT fluorescence responses induced by these controls were slower and weaker than those elicited by tissue-matched ALS specimens. The accuracy of our SOD1 RT-QuIC assay was evaluated by establishing a threshold (see methods) to determine sensitivity and specificity using receiver operating characteristic curves or ROC plots. FIG. 47 shows two ROC plots for all cervical and thoracic spinal cord data at 10⁻³ and 10⁻⁴ dilutions, and, using highest likelihood ratios, sensitivity, specificity, and area under ROC curve are 0.688, 0.938, and 0.861 for 10⁻³ dilutions, and 0.742, 0.806, and 0.833 for 10⁻⁴ dilutions, respectively (Table 4).

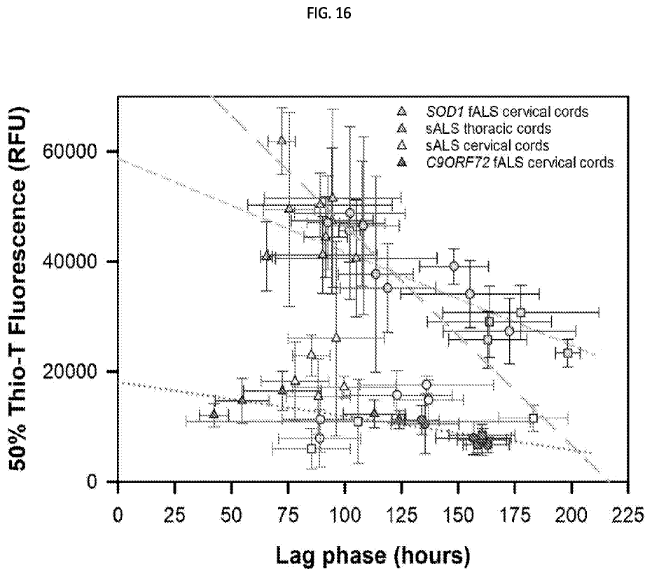

The negative control spinal cords (FIG. 19) at these dilutions failed to elicit ThT fluorescence above 10,000 RFUs until ~200 hours. Thus, lag phase and 50% ThT fluorescence are strongly negatively correlated in these ALS patient spinal cords, which likely is dependent on SOD1 seed concentration in the tissue (Table 2 and FIG. 16).

TABLE 2
ALS patient spinal cord SOD1 RT-QuIC parameters (mean ± standard deviation)

| | Lag phase (hour) | | | ThT half max (RFU) | | |
|---|---|---|---|---|---|---|
| ALS patient | 10 [symbol] | 10 [symbol] | 10 [symbol] | 10 [symbol] | 10 [symbol] | 10 [symbol] |
| SOD1 fALS patient 1 | 91.5 ± 9 | 172 ± 29 | — | 44,463 ± 7,307 | 27,348 ± 5,979 | — |
| SOD1 fALS patient 2 | 64.6 ± 2 | 76.5 ± [symbol] | 177 ± [symbol] | 40,939 ± 6,273 | 45,659 ± 5,748 | 30,000 ± 4,962 |
| SOD1 fALS patient 3 | 90.2 ± 24 | 155 ± 30 | 163 ± 27 | 41,200 ± 6,964 | 14,100 ± 6,131 | 29,100 ± 6,502 |
| SOD1 fALS patient 4 | 94.2 ± 18 | 202 ± 18 | 196 ± 5 | 47,439 ± 13,233 | 26,523 ± 8,298 | 17,521 ± 11,860 |
| SOD1 fALS patient 5 | 104 ± 35 | 148 ± 15 | 163 ± 17 | 40,600 ± 10,623 | 19,100 ± 3,191 | 25,809 ± 5,144 |
| sALS patient 1 | 99.7 ± 22 | 122 ± 24 | 183 ± 15 | 17,161 ± 1,958 | 15,708 ± 4,449 | 11,533 ± 2,373 |
| sALS patient 2 | 18 ± 15 | 89 ± 18 | 85 ± 17 | 18,215 ± 7,152 | 7,854 ± 3,189 | [symbol] ± 3,661 |
| sALS patient 3 | 96 ± 21 | 136 ± 15 | — | 26,024 ± 17,685 | 14,834 ± 3,631 | — |
| sALS patient 4 | 88 ± 14 | 89 ± 16 | [symbol] ± 75 | 15,472 ± 6,820 | 11,269 ± 5,886 | 10,974 ± 7,683 |
| sALS patient 5 | 85 ± 8 | 136 ± 29 | — | 22,948 ± [symbol] | 17,594 ± 1,571 | — |
| sALS patient 6* | 72.1 ± 6 | 102 ± 24 | — | 61,900 ± 6,053 | 48,800 ± 15,707 | — |
| sALS patient 7* | 89 ± 31 | 108 ± 15 | — | 50,300 ± 5,835 | 46,500 ± 16,140 | — |
| sALS patient 8* | 107 ± 10 | 118 ± 21 | — | 46,900 ± 11,332 | [symbol] ± 8,046 | — |
| sALS patient 9* | 75 ± 11 | 113 ± 16 | — | 49,500 ± 17,642 | 37,700 ± 17,796 | — |
| sALS patient 10* | 94 ± 30 | 92 ± 9 | — | 51,500 ± 16,218 | 47,100 ± [symbol] | — |
| C9ORF72 fALS patient 1 | 123 ± [symbol] | 163 ± 9 | 158 ± 6.4 | 11,300 ± 1,621 | 6,710 ± 1,377 | 6,720 ± 1,468 |
| C9ORF72 fALS patient 2 | 113 ± 13 | 156 ± 16 | — | 12,300 ± 2,496 | 7,940 ± 3,015 | — |
| C9ORF72 fALS patient 3 | 72.5 ± 17 | 135 ± 15 | 160 ± 10 | 16,300 ± 3,545 | 10,500 ± 5,432 | 7,610 ± 2,820 |
| C9ORF72 fALS patient 4 | 42.2 ± 6 | 133 ± 7 | 160 ± 14 | 12,100 ± 2,127 | 11,200 ± 2,614 | 8,380 ± 1,268 |
| C9ORF72 fALS patient 5 | [symbol] ± 11 | — | — | 14,700 ± [symbol] | — | — |

*Thoracic spinal cord [symbol]
indicates data missing or illegible when filed

TABLE 3
SOD1 RT-QuIC parameters (mean ± standard deviation) on patient neural tissue.

| | Lag phase (hour) | | | 50% ThT fluorescence (RFU) | | |
|---|---|---|---|---|---|---|
| | 10 [symbol] | 10 [symbol] | 10 [symbol] | 10 [symbol] | 10 [symbol] | 10 [symbol] |
| ALS patient | | | | | | |
| SOD1 fALS patient 1\hat{ } | 91.5 ± 9 | 172 ± 29 | — | 44,463 ± 7,307 | 27,348 ± 5,979 | — |
| SOD1 fALS patient 2\hat{ } | 64.6 ± 2 | 76.5 ± 51 | 177 ± 34 | 40,939 ± 6,273 | 45,659 ± 5,748 | 30,000 ± 4,962 |
| SOD1 fALS patient 3\hat{ } | 90.2 ± 24 | 155 ± 30 | 163 ± 27 | 41,200 ± 6,964 | 34,100 ± 6,131 | 29,100 ± 6,502 |
| SOD1 fALS patient 4\hat{ } | 94.2 ± 18 | 202 ± 18 | 196 ± 5 | 47,439 ± 13,233 | 26,523 ± 8,298 | 17,521 ± 11,860 |
| SOD1 fALS patient 5\hat{ } | 104 ± 35 | 148 ± 15 | 163 ± 17 | 40,600 ± 10,623 | 39,100 ± 3,193 | 25,809 ± 5,144 |
| sALS patient 1 [symbol] | 99.7 ± 22 | 122 ± 24 | 183 ± 15 | 17,161 ± 1,958 | 15,708 ± 4,449 | 11,533 ± 2,373 |
| sALS patient 2 [symbol] | 78 ± 15 | 89 ± 18 | 85 ± 17 | 18,215 ± 7,152 | 7,854 ± 5,189 | 5,997 ± 3,661 |
| sALS patient 3 [symbol] | 96 ± 21 | 136 ± 15 | — | 26,024 ± 17,685 | 14,834 ± 3,631 | — |
| sALS patient 4 [symbol] | 88 ± 14 | 89 ± 16 | 105 ± 75 | 15,472 ± 6,820 | 11,269 ± 5,886 | 10,974 ± 7,683 |
| sALS patient 5 [symbol] | 85 ± 8 | 136 ± 29 | — | 22,948 ± 3,751 | 17,594 ± 1,571 | — |
| sALS patient 6* | 72.1 ± 6 | 102 ± 24 | — | 61,900 ± 6,053 | 48,800 ± 15,707 | — |
| sALS patient 6 [symbol] | — | 88.7 ± 12 | 112 ± 0.67 | — | 20,400 ± 9,856 | 13,672 ± 797 |
| sALS patient 7* | 89 ± 31 | 108 ± 15 | — | 50,300 ± 5,835 | 46,500 ± 16,140 | — |
| sALS patient 7 [symbol] | — | 65.8 ± 11 | 101 ± 7.2 | — | 24,100 ± 11,787 | 19,793 ± 4,916 |
| sALS patient 8* | 107 ± 10 | 118 ± 21 | — | 46,900 ± 11,332 | 35,200 ± 8,046 | — |
| sALS patient 8 [symbol] | — | 75.3 ± 13 | 84.5 ± 8.1 | — | 24,700 ± 5,816 | 22,370 ± 5,226 |
| sALS patient 9* | 75 ± 11 | 113 ± 16 | — | 49,500 ± 17,642 | 37,700 ± 17,796 | — |
| sALS patient 9 [symbol] | — | 73.2 ± 29 | 104.5 ± 26 | — | 17,852 ± 8,750 | 13,956 ± 4,311 |
| sALS patient 10* | 94 ± 30 | 92 ± 9 | — | 51,500 ± 16,218 | 47,100 ± 8,480 | — |
| sALS patient 10 [symbol] | — | 57.8 ± 7 | 134.4 ± 67 | — | 25,500 ± 8,735 | 18,596 ± 13,708 |
| C9ORF72 fALS patient 1 [symbol] | 123 ± 3 | 163 ± 9 | 158 ± 6.4 | 11,300 ± 1,621 | 6,710 ± 1,377 | 6,720 ± 1,468 |
| C9ORF72 fALS patient 2 [symbol] | 113 ± 13 | 156 ± 16 | — | 12,300 ± 2,496 | 7,940 ± 3,015 | — |
| C9ORF72 fALS patient 3 [symbol] | 72.5 ± 17 | 135 ± 15 | 160 ± 10 | 16,500 ± 3,545 | 10,500 ± 5,432 | 7,610 ± 2,820 |
| C9ORF72 fALS patient 4 [symbol] | 42.2 ± 6 | 133 ± 7 | 160 ± 14 | 12,100 ± 2,127 | 11,200 ± 2,614 | 8,380 ± 1,268 |
| C9ORF72 fALS patient 5 [symbol] | 54.6 ± 11 | — | — | 14,700 ± 4,086 | — | — |
| non-ALS patient | | | | | | |
| Negative control 6 | 130 ± 10 | 159 ± 42 | 194 ± 46 | 49,400 ± 9,833 | 39,700 ± 15,683 | 23,700 ± 17,605 |
| Negative control 7 | 170 ± 16 | 203 ± 22 | — | 49,800 ± 14,864 | 30,000 ± 5,995 | — |
| Negative control 8 | 110 ± 23 | 141 ± 12 | 152 ± 7 | 6,000 ± 820 | 4,082 ± 852 | 3,120 ± 1,071 |
| Negative control 8 | 148 ± 19 | 158 ± 13 | 164 ± 16 | 4,076 ± 2,942 | 6,520 ± 1,763 | 7,140 ± 1,197 |
| Negative control 9 | 150 ± 34 | 192 ± 41 | 172 ± 18 | 41,400 ± 37,744 | 69,600 ± 85,742 | 14,100 ± 2,314 |
| Negative control 9 | 144 ± 16 | 162 ± 17 | 156 ± 3.2 | 8,254 ± 2,851 | 8,390 ± 3,996 | 5,216 ± 947 |
| Negative control 10 | 176 ± 17 | 172 ± 5 | 161 ± 17 | 15,000 ± 17,166 | 20,278 ± 18,623 | 12,862 ± 13,602 |
| Negative control 10 | 106 ± 23 | 143 ± 4 | 144 ± 20 | 20,568 ± 694 | 19,314 ± 2,415 | 17,379 ± 3,750 |
| Negative control 11 | 99 ± 19 | 150 ± 3 | 133 ± 4 | 7,464 ± 1,785 | 3,770 ± 226 | 3,215 ± 665 |
| Negative control 12 | 120 ± 12 | 158 ± 21 | 128 ± 5 | 22,474 ± 2,299 | 15,113 ± 4,335 | 18,699 ± 405 |

[symbol]

CSC,
*TSC, [symbol]
MCx [symbol]
indicates data missing or illegible when filed

TABLE 4
ROC curve parameters for 20 ALS and 12 non-ALS spinal cords.

| | | | | | |
|---|---|---|---|---|---|
| Area under ROC curve | sensitivity | 95% CI | specificity | 95% CI | likelihood ratio |

10⁻³ spinal cord dilutions

| | | | | | | | |
|---|---|---|---|---|---|---|---|
| Area | 0.861 | >0.125 | 0.781 | 0.612-0.890 | 0.844 | 0.682-0.931 | 5.0 |
| std error | 0.0493 | >0.375 | 0.688 | 0.514-0.820 | 0.938 | 0.799-0.989 | 11.0 |
| 95% CI | 0.765-0.958 | >0.75 | 0.625 | 0.453-0.771 | 1.00 | 0.893-1.0 | — |
| p value | <0.0001 | | | | | | |

10⁻⁴ spinal cord dilutions

| | | | | | | | |
|---|---|---|---|---|---|---|---|
| Area | 0.833 | >0.125 | 0.839 | 0.674-0.929 | 0.548 | 0.378-0.708 | 1.86 |
| std error | 0.0536 | >0.375 | 0.742 | 0.568-0.863 | 0.806 | 0.637-0.908 | 3.83 |
| 95% CI | 0.728-0.938 | >0.625 | 0.548 | 0.378-0.708 | 1.00 | 0.89-1.0 | — |
| p value | <0.0001 | >0.875 | 0.387 | 0.237-0.562 | 1.00 | 0.89-1.0 | — |

Transmission Electron Microscopy of SOD1 RT-QuIC Products from ALS Patient Spinal Cords

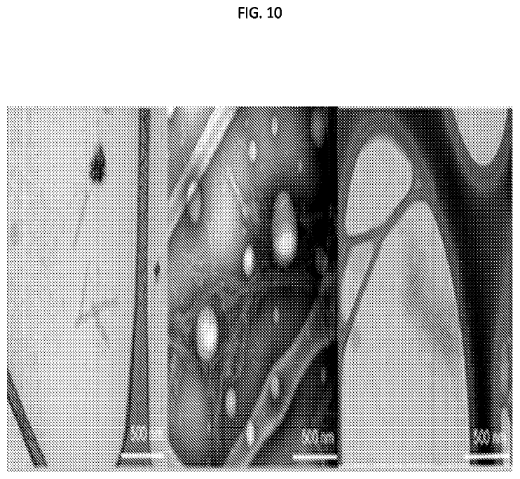

After converted SOD1 RT-QuIC ALS samples reached stationary phase, the converted (ThT positive) or non-converted (ThT negative) wells were scraped, pooled at 10⁻³ dilution, and imaged on a transmission electron microscope (see methods). Samples that were considered ThT positive by SOD1 RT-QuIC depicted several amyloid fibrils relative to negative control samples (FIG. 10), suggesting that the described assay may successfully detect and propagate misfolded forms of SOD1 in human SOD1 fALS and sALS spinal cords. Based on the size of these aggregates with the scale (See FIG. 10), the SOD1 RT-QuIC assay may propagate different sizes and possibly morphologies of SOD1 aggregates.

### Example 2—SOD1 RT-QuIC Protocol for CSF

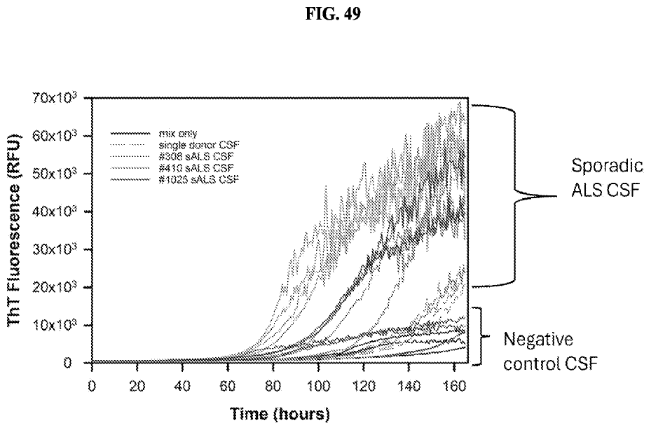

The protocol used to generate the data in FIGS. 49-51 is the same as described in herein, except the final SOD1 substrate concentration used in the assay is 30 micromole/liter (uM) using extinction coefficient described herein. CSF is diluted 1:10 (18 microliters water to 2 microliters of CSF) in high performance liquid chromatography water (Sigma Aldrich, #270733-4 L). Wells are seeded with 1:10 dilutions of CSF. The plate is inserted in a BMG Labtech with settings described in herein (SOD1 RT-QuIC assay, Paragraph [0148]). Data analysis is done as described in Paragraphs [0171]-[0176]. ALS patient clinical data (ALS Functional Rating Scale Revised (ALSFRS-R) slope decline, age, sex, time of CSF collection in disease) was provided by Dr. Cindy Ly (Washington University in St. Louis).

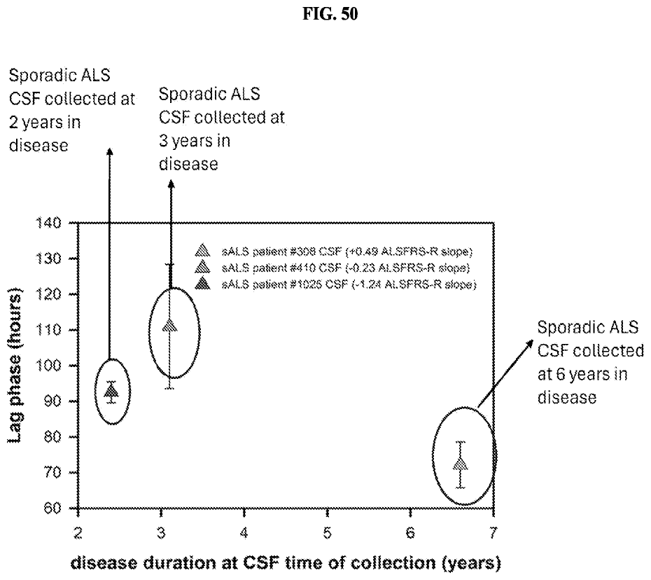

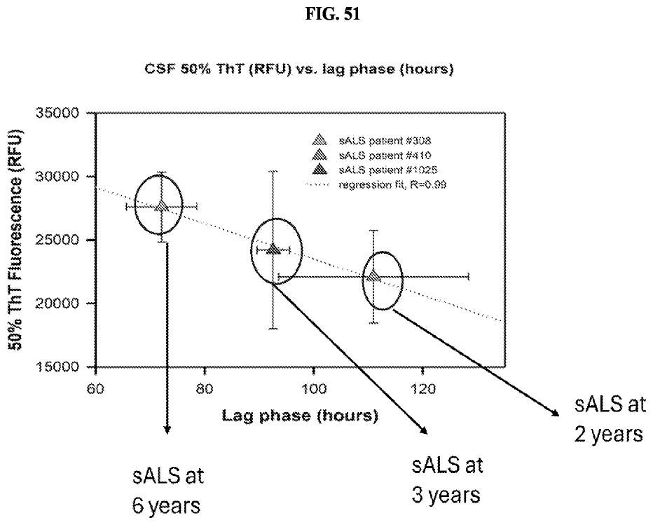

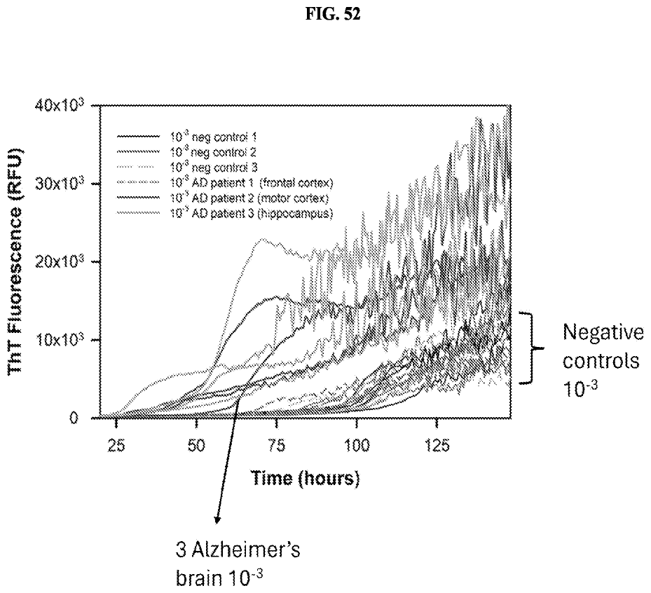

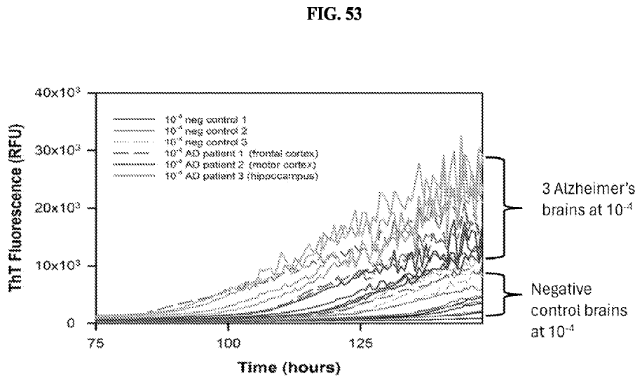

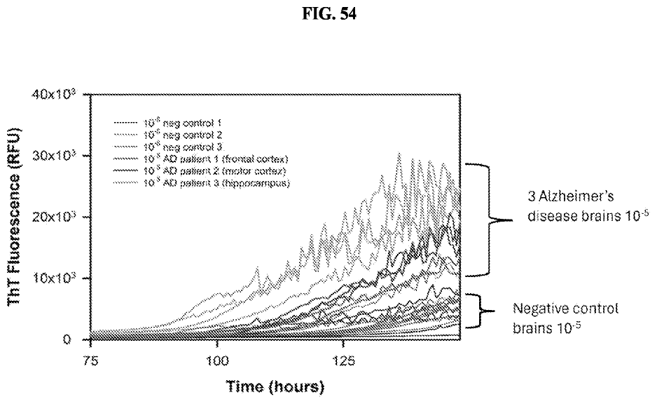

Table 5 below shows sporadic ALS patient sex, age, and their ALS functional rating scale revised (ALSFRS-R) slope decline shown in FIG. 49, which is a measure of how quickly an ALS patient is progressing with their disease (more negative the slope, the faster the progression). The time CSF was collected in their disease, and SOD1 RT-QuIC kinetic parameters are also shown. In FIG. 50, lag phase in Table 5 is plotted versus disease duration, and in FIG. 51, 50% ThT (RFU) is plotted versus lag phase, with a strong negative correlation (R=0.99). FIGS. 50 and 51 indicate lag phase correlates with ALS disease progression, the shorter the lag phase, the more SOD1 seeding activity and longer a patient has had ALS.

TABLE 5
SOD1 seeding activity correlates with sporadic
ALS disease progression via lag phase.

| | | | | Disease | | |
|---|---|---|---|---|---|---|
| | | | | duration | | |
| | | | | at time | SOD1 | SOD1 |
| sporadic | | | | of CSF | RT- | RT-QuIC |
| ALS | | | ALSFRS- | collec- | QuIC | 50% ThT |
| patient | | age | R | tion | lag | Fluorescence |
| ID | sex | (years) | decline | (years) | phase | (RFU) |
| #308 | F | 67.35 | 0.4908 | 6.63 | 72.1 ± 6.4 | 27,600 ± 2,746 |
| #410 | F | 56.01 | −0.2362 | 3.11 | 110 ± 17 | 22,100 ± 3,655 |
| #1025 | F | 73.63 | −1.2429 | 2.40 | 92.5 ± 2.9 | 24,200 ± 6,202 |

### Example 3

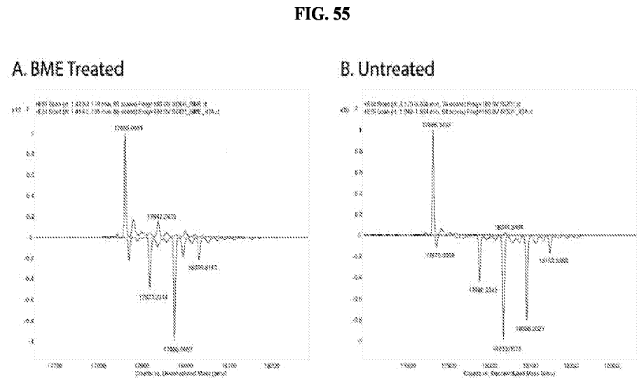

βME (beta mercaptoethanol)-treated SOD1 was prepared as described herein in Paragraph [0161]. Five microliters of 2% Sodium dodecyl sulfate and 45 microliters of 200 millimolar (mM) ammonium bicarbonate (pH 8.0) were added to 20 micrograms of βME-treated SOD1 protein sample. Volume was slightly adjusted with ultrapure water for final iodoacetamide (IOA) concentration of 15 millimolar (ThermoFisher Scientific, A39271) at pH 8.0 for alkylation in the dark for 30 minutes prior to protein mass determination by electrospray ionization mass spectrometry (ESI-MS). A trypsin digest was performed before ESI-MS analysis to determine free cysteine residues in SOD1 substrate treated with βME. Samples were analyzed using an Agilent 6520 Q-TOF mass spectrometer coupled to an Agilent 1260 UPLC equipped with a AdvancedBio SEC column. IOA adds a +57 mass signature to free Cysteine residues. The deconvoluted protein mass spectra in FIG. 55 shows the predominant mass of 17,980 Daltons, indicative of 2 labeled Cysteine residues. FIG. 55B shows untreated SOD1 incubated with IOA results in all four Cysteine residues being labeled, indicative of no disulfide bonds. Results (see, FIG. 55) suggest the single intramolecular disulfide bond in βME treated SOD1 are Cysteine residues 75 and 164 in SEQ ID NO:3, while Cysteine residues 24 and 129 are free thiols (no disulfide bond).

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure and should not be viewed as limiting to the scope of the disclosure. The disclosures of all journal references, U.S. patents, and publications referred to herein are hereby incorporated by reference in their entireties.

The present disclosure has multiple aspects, illustrated by the non-limiting examples described herein.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A method of preparing a purified human Superoxide Dismutase 1 (SOD1) substrate for use in a real-time quaking induced conversion assay, the method comprising:
a. performing chromatography on a sample of cells expressing a human SOD1 protein having an amino acid sequence of SEQ ID NO:1 or SEQ ID NO:2, to produce an eluate; and
b. either:
i. incubating the eluate for at least 6 hours at a temperature of about 20° C. to about 22° C. in at least 20 mM to about 30 mM of at least one buffer having a pH between about 6.0 to about 8.0 to produce a purified human SOD1 substrate; or
ii. performing, in the presence of at least 20 mM to about 30 mM of at least one buffer having a pH between about 6.0 to about 8.0, buffer exchange, exchange using an exclusion column which removes any salts and/or contaminants less than 10 kDa, or dialysis for at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours to produce a purified human SOD1 substrate.

2. The method of claim 1, wherein the method further comprises: (a) centrifuging the eluate after incubation to obtain a supernatant; and (b) filtering the supernatant to produce a purified human SOD1 substrate.

3. The method of claim 1 or claim 2, wherein the chromatography is liquid chromatography, ion exchange chromatography, affinity chromatography, size exclusion chromatography, or any combinations thereof.

4. The method of any of claims 1-3, wherein the buffer is a Tris buffer, a sodium phosphate buffer, HEPES, Tris-HCl, or any combinations thereof.

5. The method of any of claims 1-4, wherein the eluate is incubated for at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

6. The method of any of claims 1-5, wherein the concentration of at least one buffer is between about 10 mM to about 90 mM.

7. The method of any of claims 1-6, wherein the pH is about 8.0.

8. A method for detecting the presence of at least one misfolded form of human Superoxide Dismutase 1 (SOD1) in a biological sample obtained from a human subject, the method comprising the steps of:
a. performing a real-time quaking induced conversion (RT-QuIC) assay or seed amplification assay (SAA) on a biological sample obtained from a subject suspected of having a neurodegenerative disease, wherein the RT-QuIC assay:
i. uses the human SOD1 substrate produced according to the method of claim 1; and
ii. is performed:
1. using a buffer comprising: (i) about 0.5 M to about 0.75 M guanidine HCl or urea; and (ii) about 0.01 M to about 0.05 M sodium acetate;
2. using at least one fluorescent compound;
3. at a pH of about 3.5 to about 4.5;
4. at a shaker speed of about 300 to about 700 rpm; and
5. at a temperature of about 35° C. to about 42° C.; and
b. detecting the presence of the at least one fluorescent compound, wherein the detection of the presence of the at least one fluorescent compound indicates the presence of at least one misfolded form of SOD1 in the biological sample.

9. The method of claim 8, wherein the buffer further comprises EDTA, one or more reducing agents, one or more salts, or any combinations thereof.

10. The method claim 8 or claim 9, wherein the concentration of human SOD1 substrate used in the assay is from about 10 μM to about 60 μM.

11. The method of claim 10, wherein the concentration of human SOD1 substrate used in the assay is about 30 μM based on an extinction coefficient of 5,500 M⁻¹ cm⁻¹.

12. The method of any of claims 8-11, wherein the buffer comprises (i) about 0.6 M guanidine HCl or urea; and (ii) about 0.02 M sodium acetate.

13. The method of claim 12, wherein the buffer further comprises EDTA, one or more reducing agents, and one or more salts.

14. The method of any of claims 8-13, wherein the pH is about 4.0.

15. The method of any of claims 8-14, wherein the shaker speed is about 500 rpm.

16. The method of any of claims 8-15, wherein the temperature is about 37° C.

17. The method of any of claims 8-16, wherein the fluorescent compound is an amyloid binding dyes.

18. The method of claim 17, wherein the concentration of fluorescent compound is from about 5 μM to about 100 μM.

19. The method of claim 18, wherein the concentration of fluorescent compound is about 20 μM.

20. The method of any of claims 8-19, wherein the biological sample is cerebrospinal fluid, tissue, whole blood, serum, plasma, saliva, nasal brushings, skin, urine, tears, or any combinations thereof.

21. The method of claim 20, wherein the tissue is spinal cord tissue, brain tissue, or any combinations thereof.

22. The method of any of claims 8-21, wherein the neurodegenerative disease is amyotrophic lateral sclerosis (ALS), Parkinson's disease, or Alzheimer's disease.

23. The method of claim 22, wherein the ALS is familial ALS or sporadic ALS.

24. The method of any of claims 8-23, wherein the method further comprises diagnosing a subject as having a neurodegenerative disease based on the detection of the presence of one or more misfolded forms of SOD1 in the biological sample obtained from the human subject.

25. The method of claim 24, wherein the method further comprises monitoring the subject for progression of the neurogenerative disease.

26. The method of claim 24 or claim 25, wherein the method further comprises administering one or more treatments to the subject diagnosed as having a neurodegenerative disease.

27. The method of any of claims 8-27, wherein the assay uses one or more solid supports.

28. The method of claim 27, wherein the solid support is one or more beads or a 96-well plate.


SEQUENCE LISTING (5 sequences)

SEQ ID NO: 1 - protein, 171 aa, synthetic construct
  REGION 1..171: note = Synthetic
MHHHHHHSSGLVPRGSHMATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 2 - protein, 154 aa, synthetic construct [sequence omitted]

SEQ ID NO: 3 - protein, 5749 aa, synthetic construct
  REGION 1..5749: note = Synthetic
TGGCGAATGGGACGCGCCCTGTAGCGGCGCATTAAGCGCGGCGGGTGTGGTGGTTACGCGCAGCGTGACCGCTACACTTGCCAGCGCCCTAGCGCCCGCTCCTTTCGCTTTCTTCCCTTCCTTTCTCGCCACGTTCGCCGGCTTTCCCCGTCAAGCTCTAAATCGGGGGCTCCCTTTAGGGTTCCGATTTAGTGCTTTACGGCACCTCGACCCCAAAAAACTTGATTAGGGTGATGGTTCACGTAGTGGGCCATCGCCCTGATAGACGGTTTTTCGCCCTTTGACGTTGGAGTCCACGTTCTTTAATAGTGGACTCTTGTTCCAAACTGGAACAACACTCAACCCTATCTCGGTCTATTCTTTTGATTTATAAGGGATTTTGCCGATTTCGGCCTATTGGTTAAAAAATGAGCTGATTTAACAAAAATTTAACGCGAATTTTAACAAAATATTAACGTTTACAATTTCAGGTGGCACTTTTCGGGGAAATGTGCGCGGAACCCCTATTTGTTTATTTTTCTAAATACATTCAAATATGTATCCGCTCATGAATTAATTCTTAGAAAAACTCATCGAGCATCAAATGAAACTGCAATTTATTCATATCAGGATTATCAATACCATATTTTTGAAAAAGCCGTTTCTGTAATGAAGGAGAAAACTCACCGAGGCAGTTCCATAGGATGGCAAGATCCTGGTATCGGTCTGCGATTCCGACTCGTCCAACATCAATACAACCTATTAATTTCCCCTCGTCAAAAATAAGGTTATCAAGTGAGAAATCACCATGAGTGACGACTGAATCCGGTGAGAATGGCAAAAGTTTATGCATTTCTTTCCAGACTTGTTCAACAGGCCAGCCATTACGCTCGTCATCAAAATCACTCGCATCAACCAAACCGTTATTCATTCGTGATTGCGCCTGAGCGAGACGAAATACGCGATCGCTGTTAAAAGGACAATTACAAACAGGAATCGAATGCAACCGGCGCAGGAACACTGCCAGCGCATCAACAATATTTTCACCTGAATCAGGATATTCTTCTAATACCTGGAATGCTGTTTTCCCGGGGATCGCAGTGGTGAGTAACCATGCATCATCAGGAGTACGGATAAAATGCTTGATGGTCGGAAGAGGCATAAATTCCGTCAGCCAGTTTAGTCTGACCATCTCATCTGTAACATCATTGGCAACGCTACCTTTGCCATGTTTCAGAAACAACTCTGGCGCATCGGGCTTCCCATACAATCGATAGATTGTCGCACCTGATTGCCCGACATTATCGCGAGCCCATTTATACCCATATAAATCAGCATCCATGTTGGAATTTAATCGCGGCCTAGAGCAAGACGTTTCCCGTTGAATATGGCTCATAACACCCCTTGTATTACTGTTTATGTAAGCAGACAGTTTTATTGTTCATGACCAAAATCCCTTAACGTGAGTTTTCGTTCCACTGAGCGTCAGACCCCGTAGAAAAGATCAAAGGATCTTCTTGAGATCCTTTTTTTCTGCGCGTAATCTGCTGCTTGCAAACAAAAAAACCACCGCTACCAGCGGTGGTTTGTTTGCCGGATCAAGAGCTACCAACTCTTTTTCCGAAGGTAACTGGCTTCAGCAGAGCGCAGATACCAAATACTGTCCTTCTAGTGTAGCCGTAGTTAGGCCACCACTTCAAGAACTCTGTAGCACCGCCTACATACCTCGCTCTGCTAATCCTGTTACCAGTGGCTGCTGCCAGTGGCGATAAGTCGTGTCTTACCGGGTTGGACTCAAGACGATAGTTACCGGATAAGGCGCAGCGGTCGGGCTGAACGGGGGGTTCGTGCACACAGCCCAGCTTGGAGCGAACGACCTACACCGAACTGAGATACCTACAGCGTGAGCTATGAGAAAGCGCCACGCTTCCCGAAGGGAGAAAGGCGGACAGGTATCCGGTAAGCGGCAGGGTCGGAACAGGAGAGCGCACGAGGGAGCTTCCAGGGGGAAACGCCTGGTATCTTTATAGTCCTGTCGGGTTTCGCCACCTCTGACTTGAGCGTCGATTTTTGTGATGCTCGTCAGGGGGGCGGAGCCTATGGAAAAACGCCAGCAACGCGGCCTTTTTACGGTTCCTGGCCTTTTGCTGGCCTTTTGCTCACATGTTCTTTCCTGCGTTATCCCCTGATTCTGTGGATAACCGTATTACCGCCTTTGAGTGAGCTGATACCGCTCGCCGCAGCCGAACGACCGAGCGCAGCGAGTCAGTGAGCGAGGAAGCGGAAGAGCGCCTGATGCGGTATTTTCTCCTTACGCATCTGTGCGGTATTTCACACCGCATATATGGTGCACTCTCAGTACAATCTGCTCTGATGCCGCATAGTTAAGCCAGTATACACTCCGCTATCGCTACGTGACTGGGTCATGGCTGCGCCCCGACACCCGCCAACACCCGCTGACGCGCCCTGACGGGCTTGTCTGCTCCCGGCATCCGCTTACAGACAAGCTGTGACCGTCTCCGGGAGCTGCATGTGTCAGAGGTTTTCACCGTCATCACCGAAACGCGCGAGGCAGCTGCGGTAAAGCTCATCAGCGTGGTCGTGAAGCGATTCACAGATGTCTGCCTGTTCATCCGCGTCCAGCTCGTTGAGTTTCTCCAGAAGCGTTAATGTCTGGCTTCTGATAAAGCGGGCCATGTTAAGGGCGGTTTTTTCCTGTTTGGTCACTGATGCCTCCGTGTAAGGGGGATTTCTGTTCATGGGGGTAATGATACCGATGAAACGAGAGAGGATGCTCACGATACGGGTTACTGATGATGAACATGCCCGGTTACTGGAACGTTGTGAGGGTAAACAACTGGCGGTATGGATGCGGCGGGACCAGAGAAAAATCACTCAGGGTCAATGCCAGCGCTTCGTTAATACAGATGTAGGTGTTCCACAGGGTAGCCAGCAGCATCCTGCGATGCAGATCCGGAACATAATGGTGCAGGGCGCTGACTTCCGCGTTTCCAGACTTTACGAAACACGGAAACCGAAGACCATTCATGTTGTTGCTCAGGTCGCAGACGTTTTGCAGCAGCAGTCGCTTCACGTTCGCTCGCGTATCGGTGATTCATTCTGCTAACCAGTAAGGCAACCCCGCCAGCCTAGCCGGGTCCTCAACGACAGGAGCACGATCATGCGCACCCGTGGGGCCGCCATGCCGGCGATAATGGCCTGCTTCTCGCCGAAACGTTTGGTGGCGGGACCAGTGACGAAGGCTTGAGCGAGGGCGTGCAAGATTCCGAATACCGCAAGCGACAGGCCGATCATCGTCGCGCTCCAGCGAAAGCGGTCCTCGCCGAAAATGACCCAGAGCGCTGCCGGCACCTGTCCTACGAGTTGCATGATAAAGAAGACAGTCATAAGTGCGGCGACGATAGTCATGCCCCGCGCCCACCGGAAGGAGCTGACTGGGTTGAAGGCTCTCAAGGGCATCGGTCGAGATCCCGGTGCCTAATGAGTGAGCTAACTTACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCCAGGGTGGTTTTTCTTTTCACCAGTGAGACGGGCAACAGCTGATTGCCCTTCACCGCCTGGCCCTGAGAGAGTTGCAGCAAGCGGTCCACGCTGGTTTGCCCCAGCAGGCGAAAATCCTGTTTGATGGTGGTTAACGGCGGGATATAACATGAGCTGTCTTCGGTATCGTCGTATCCCACTACCGAGATATCCGCACCAACGCGCAGCCCGGACTCGGTAATGGCGCGCATTGCGCCCAGCGCCATCTGATCGTTGGCAACCAGCATCGCAGTGGGAACGATGCCCTCATTCAGCATTTGCATGGTTTGTTGAAAACCGGACATGGCACTCCAGTCGCCTTCCCGTTCCGCTATCGGCTGAATTTGATTGCGAGTGAGATATTTATGCCAGCCAGCCAGACGCAGACGCGCCGAGACAGAACTTAATGGGCCCGCTAACAGCGCGATTTGCTGGTGACCCAATGCGACCAGATGCTCCACGCCCAGTCGCGTACCGTCTTCATGGGAGAAAATAATACTGTTGATGGGTGTCTGGTCAGAGACATCAAGAAATAACGCCGGAACATTAGTGCAGGCAGCTTCCACAGCAATGGCATCCTGGTCATCCAGCGGATAGTTAATGATCAGCCCACTGACGCGTTGCGCGAGAAGATTGTGCACCGCCGCTTTACAGGCTTCGACGCCGCTTCGTTCTACCATCGACACCACCACGCTGGCACCCAGTTGATCGGCGCGAGATTTAATCGCCGCGACAATTTGCGACGGCGCGTGCAGGGCCAGACTGGAGGTGGCAACGCCAATCAGCAACGACTGTTTGCCCGCCAGTTGTTGTGCCACGCGGTTGGGAATGTAATTCAGCTCCGCCATCGCCGCTTCCACTTTTTCCCGCGTTTTCGCAGAAACGTGGCTGGCCTGGTTCACCACGCGGGAAACGGTCTGATAAGAGACACCGGCATACTCTGCGACATCGTATAACGTTACTGGTTTCACATTCACCACCCTGAATTGACTCTCTTCCGGGCGCTATCATGCCATACCGCGAAAGGTTTTGCGCCATTCGATGGTGTCCGGGATCTCGACGCTCTCCCTTATGCGACTCCTGCATTAGGAAGCAGCCCAGTAGTAGGTTGAGGCCGTTGAGCACCGCCGCCGCAAGGAATGGTGCATGCAAGGAGATGGCGCCCAACAGTCCCCCGGCCACGGGGCCTGCCACCATACCCACGCCGAAACAAGCGCTCATGAGCCCGAAGTGGCGAGCCCGATCTTCCCCATCGGTGATGTCGGCGATATAGGCGCCAGCAACCGCACCTGTGGCGCCGGTGATGCCGGCCACGATGCGTCCGGCGTAGAGGATCGAGATCTCGATCCCGCGAAATTAATACGACTCACTATAGGGGAATTGTGAGCGGATAACAATTCCCCTCTAGAAATAATTTTGTTTAACTTTAAGAAGGAGATATACCATGCATCATCATCATCATCACAGCAGCGGCCTGGTGCCGCGCGGCAGCCATATGGCAACAAAAGCTGTATGTGTTCTAAAGGGCGATGGTCCGGTTCAAGGTATCATCAACTTCGAGCAGAAAGAATCTAATGGCCCGGTCAAGGTGTGGGGTTCGATCAAAGGTTTGACCGAAGGTTTACATGGTTTTCATGTTCACGAGTTCGGCGACAACACCGCGGGCTGCACGTCTGCAGGCCCACACTTTAACCCGCTGTCCCGCAAGCACGGCGGTCCGAAAGATGAAGAACGTCATGTGGGTGACCTGGGCAATGTTACTGCGGACAAGGACGGCGTGGCCGACGTTAGCATTGAAGATAGCGTTATTAGCCTGAGCGGTGACCACTGCATTATTGGTCGTACCTTGGTCGTGCACGAGAAGGCGGATGATCTGGGTAAAGGCGGAAACGAGGAGTCCACCAAAACCGGTAATGCTGGCAGCCGTCTGGCATGTGGTGTGATCGGCATCGCGCAGTAACTCGAGCACCACCACCACCACCACTGAGATCCGGCTGCTAACAAAGCCCGAAAGGAAGCTGAGTTGGCTGCTGCCACCGCTGAGCAATAACTAGCATAACCCCTTGGGGCCTCTAAACGGGTCTTGAGGGGTTTTTTGCTGAAAGGAGGAACTATATCCGGAT

SEQ ID NO: 4 - protein, 171 aa, synthetic construct; identical to SEQ ID NO: 1
  REGION 1..171: note = Synthetic

SEQ ID NO: 5 - protein, 5749 aa, synthetic construct; identical to SEQ ID NO: 3
  REGION 1..5749: note = Synthetic